(19) United States
(12) Patent Application Publication
(10) Pub. No.: US 2024/0400757 A1
(43) Pub. Date: Dec. 5, 2024

(54) METHOD FOR PRODUCING FLUORINATED POLYETHER

(71) Applicant: Resonac Corporation, Tokyo (JP)

(72) Inventors: Masataka UMETANI, Minato-ku, Tokyo (JP); Nobuo OSAWA, Minato-ku, Tokyo (JP); Tsuyoshi KATO, Minato-ku, Tokyo (JP); Takuma KURODA, Minato-ku, Tokyo (JP)

(73) Assignee: Resonac Corporation, Tokyo (JP)

(21) Appl. No.: 18/698,860

(22) PCT Filed: Oct. 7, 2022
(86) PCT No.: PCT/JP2022/037644

§ 371 Date: Apr. 5, 2024

(30) Foreign Application Priority Data
Oct. 8, 2021 (JP) 2021-166426

(51) Int. Cl. C08G 65/00; C08G 65/48

(52) U.S. Cl. CPC C08G 65/007; C08G 65/48

(57) ABSTRACT

To provide a method capable of producing a fluorinated polyether with a number-average molecular weight as theoretically calculated from a number-average molecular weight of a raw material polyether compound in high yield.

A method for producing a fluorinated polyether, characterized by including: a step (1) of performing an operation of adjusting a molecular weight distribution of a polymeric compound having a structural unit (R₁—O) to obtain the raw material compound represented by a formula (X); and after the step (1), a step (2) of introducing the raw material compound represented by the formula (X), an inert gas, a fluorine gas, and a solvent into a reactor to fluorinate the raw material compound, R₄—O—(R₁—O)ₓ—R₅ (X) wherein R₁ represents a divalent hydrocarbon group having 2 to 5 carbon atoms; R₄ and R₅ each independently represent a protecting group for a hydroxyl group; x represents an average degree of polymerization and is a real number of 2.7 to 15.

### TECHNICAL FIELD

This invention relates to a novel method for producing a fluorinated polyether.

### BACKGROUND ART

Perfluoropolyether compounds that are fluorinated polyethers, have been known to exhibit high performance over an extremely wide range of applications as lubricants. For this reason, the perfluoropolyether compounds are widely used in vacuum pump oils as lubricant oils, heat transfer media, non-adhesives, and other applications.

Perfluoropolyether compounds are produced by fluorinating a CH group in a raw hydrocarbon compound to a CF group, and a method of electrochemical fluorine substitution by using hydrogen fluoride (electrolytic fluorination reaction) and fluorination using a fluorine gas, have been known.

However, the electrolytic fluorination reaction has the problem that a desired compound cannot be obtained with high purity. As reaction using a fluorine gas, a vapor phase method and a liquid phase method have been known, and the vapor phase method has had the problem of causing cleavage of a C—C single bond upon reaction with a fluorine gas in a vapor phase, resulting in generating many kinds of by-products.

The liquid phase method is a method for solving such problems, and for example, Patent Literatures 1, 2 have been reported.

Patent Literature 1 discloses a method allowing liquid-phase fluorine substitution of a wide range of hydrogen-containing compounds to complete fluorine substitution. Specifically, the patent literature discloses that the hydrogen-containing compound is dissolved or dispersed in a medium such as liquid perfluorocarbon, and a mixture of fluorine gas and dilution gas is introduced for continuous fluorine substitution.

Patent Literature 2 discloses a method for using a polyether compound as a raw material, introducing a hydrogen fluoride scavenger, an inert gas, a fluorine gas, and a solvent that is a fully halogen-substituted saturated compound having 2 to 8 carbon atoms into a reactor, removing the hydrogen fluoride scavenger from the reactor, distributing a perhalogenated unsaturated compound into the reactor while distributing the inert gas and fluorine gas, to fluorinate a prescribed polyether compound.

### CITATION LIST

### Patent Literature

[Patent Literature 1] JP2945693B
[Patent Literature 2] JP6850595B

### SUMMARY OF INVENTION

### Technical Problem

However, the production method of Patent Literature 1 has had the problem that it is not suitable for fluorination of high molecular weight polyethylene glycol, is not able to yield a target product, and has low production efficiency.

In the liquid phase method, a polyether compound synthesized by sequential polymerization has also been used as a raw material. In the liquid phase method, as a hydrogen atom of a raw material compound is usually substituted with a fluorine atom, a structure of a perfluoropolyether compound to be produced can also be predicted from a structure of the raw material polyether compound.

However, even the liquid phase method has had a problem of discrepancy between a theoretical value of a number-average molecular weight calculated from a number-average molecular weight of a raw material and a number-average molecular weight of a perfluoropolyether compound that is produced, and also a reduced yield of the perfluoropolyether compound. Discrepancy between the theoretical value of the number-average molecular weight calculated from the number-average molecular weight of the raw material and the number-average molecular weight of the perfluoropolyether compound that is produced makes it difficult to produce a perfluoropolyether compound with a desired number-average molecular weight according to intended applications. The large discrepancy between the theoretical value and the measured value of the number-average molecular weight or the low yield of the perfluoropolyether compound is assumed to be derived from a large amount of components contained in the raw material, which cannot be collected even after fluorination. An expensive fluorine gas is consumed for fluorination of such components in the raw materials, resulting in high production cost.

Thus, an object of the present invention is to provide a method capable of producing a fluorinated polyether with a number-average molecular weight as theoretically calculated from a number-average molecular weight of a raw material polyether compound in high yield.

### Solution to Problem

The present inventors investigated the cause in order to solve the aforementioned problem and have found that a polyether compound used as a fluorination raw material has a wide molecular weight distribution, a low molecular weight component that has been fluorinated has a low boiling point and flows out of a system with gas introduced during reaction, while a high molecular weight component that has been fluorinated precipitates as an insoluble solid and cannot be collected, resulting also in low yield. Such reduction in yield is also a factor that increases production cost. For example, when a polyether compound used as a fluorination raw material is synthesized by a known method such as sequential polymerization, a molecular weight distribution (also called polydispersity and represented by Mw/Mn) is hardly controlled, and a molecular weight distribution of the polyether compound tends to become wider.

Then, the present inventors have found that by preliminarily adjusting a molecular weight distribution of a polyether compound used as a fluorination raw material, a low molecular weight component, for example, which causes a discrepancy between a theoretical value of a number-average molecular weight calculated from a number-average molecular weight of the raw material and a number-average molecular weight of a fluorinated polyether that is generated, can be removed, thereby enabling production of a fluorinated polyether with a number-average molecular weight as theoretically calculated from the number-average molecular weight of the raw material in high-yield.

Configurations of the present invention are as follows:

[1] A method for producing a fluorinated polyether, comprising
a step (1) of performing an operation of adjusting a molecular weight distribution of a polymeric compound having a structural unit (R₁—O) to obtain a raw material compound represented by a formula (X); and
after the step (1), a step (2) of introducing the raw material compound represented by the formula (X), an inert gas, a fluorine gas, and a solvent into a reactor to fluorinate the raw material compound.

R₄—O—(R₁—O)ₓ—R₅ (X)

wherein R₁ represents a divalent hydrocarbon group having 2 to 5 carbon atoms; R₁ in each structural unit represented by (R₁—O) may all be the same, or may be partially or totally different from each other; R₄ and R₅ each independently represent a protecting group for a hydroxyl group; and x represents an average degree of polymerization and is a real number of 2.7 to 15.

[2] The method for producing a fluorinated polyether according to [1], wherein the step (1) comprises a step (1A) of adjusting a molecular weight distribution of a compound represented by a formula (A).

R₂—O—(R₁—O)ₚ—R₃ (A)

wherein R₁ is the same as defined for the formula (X); R₂ and R₃ each independently represent a protecting group for a hydroxyl group; p represents an average degree of polymerization and is a real number of 1 or greater.

[3] The method for producing a fluorinated polyether according to [1], wherein the step (1) comprises a step (1B) of adjusting a molecular weight distribution of a compound represented by a formula (B).

HO—(R₁—O)_{q}—H (B)

wherein R₁ is the same as defined for the formula (X); q represents an average degree of polymerization and is a real number of 1 or greater.

[4] The method for producing a fluorinated polyether according to [3], wherein the step (1) comprises, after the step (1B), a step (1C) of protecting a hydroxyl group of the compound obtained by the step (1B).

[5] The method for producing a fluorinated polyether according to [4], wherein the step (1) comprises, after the step (1C), a step (1D) of adjusting a molecular weight distribution of the compound obtained by the step (1C).

[6] The method for producing a fluorinated polyether according to any one of [1] to [5], wherein the operation of adjusting a molecular weight distribution in the step (1) is at least one operation selected from:
an operation of adjusting a molecular weight distribution of a polymeric compound having the same structural unit as that in the formula (X) so as to become narrower; and
an operation of mixing two or more types of monodisperse polymers of a polymeric compound having the same structural unit as that in the formula (X) to adjust molecular weight distribution.

[7] The method for producing a fluorinated polyether according to any one of [1] to [6], wherein
the operation of adjusting a molecular weight distribution in the step (1) is an operation of adjusting a molecular weight distribution of a polymeric compound having the same structural unit as that in the formula (X) so as to become narrower.

[8] The method for producing a fluorinated polyether according to any one of [1] to [7], wherein Mw/Mn representing a molecular weight distribution of the raw material compound is 1.30 or smaller.

[9] The method for producing a fluorinated polyether according to any one of [1] to [8], wherein a total proportion of a compound represented by a formula (X-1) contained in the raw material compound is 5% or less in terms of peak area ratio based on GPC analysis.

R₄—O—(R₁—O)ᵣ—R₅ (X-1)

wherein R₁, R₄, R₅ are the same as defined for the formula (X) and r represents 1 or 2.

[10] The method for producing a fluorinated polyether according to any one of [1] to [9], wherein a total proportion of a compound represented by a formula (X-2) contained in the raw material compound is 15% or less in terms of peak area ratio based on GPC analysis.

R₄—O—(R₁—O)₃—R₅ (X-2)

wherein R₁, R₄, and R₅ are the same as defined for the formula (X), s is an integer and satisfies s≥(an average degree of polymerization x in the formula (X)+4).

[11] The method for producing a fluorinated polyether according to any one of [1] to [10], comprising, after the step (2), a step (3) of introducing a perhalogenated unsaturated hydrocarbon compound into the reactor while distributing an inert gas and a fluorine gas in the reactor.

[12] The method for producing a fluorinated polyether according to any one of [1] to [11], wherein the raw material compound is a compound in which R₄ and R₅ in the formula (X) are acyl groups.

[13] A method for producing a compound represented by a formula (Y), comprising a step (4) of reacting the fluorinated polyether obtained by the production method according to any one of [1] to [12] with an alcohol having 1 to 3 carbon atoms.

R₆O—(C═O)—Rf₂—O—(Rf₁—O)_{y}—Rf₃—(C═O)—OR₇ (Y)

wherein Rf₁ represents a divalent perfluorohydrocarbon group having 2 to 5 carbon atoms; Rf₁ in each structural unit represented by (Rf₁—O) may all be the same, or may be partially or totally different from each other; Rf₂ and Rf₃ each independently represent a perfluorohydrocarbon group having 1 to 4 carbon atoms and are determined according to a structure of a structural unit arranged at an end in the formula (X); R₆ and R₇ each independently represent an alkyl group having 1 to 3 carbon atoms; and y represents an average degree of polymerization and is a real number of 0.7 to 13.

[14] A method for producing a compound represented by a formula (Z), comprising a step (5) of reducing esters at both ends of the compound represented by the formula (Y) obtained by the production method according to [13].

HO—CH₂—Rf₂—O—(Rf₁—O)_{y}—Rf₃—CH₂—OH (Z)

wherein Rf₁, Rf₂, Rf₃, and y are the same as defined for the formula (Y).

### Advantageous Effect of Invention

According to the present invention, a fluorinated polyether with a number-average molecular weight as theoretically calculated from a number-average molecular weight of a raw material polyether compound, can be produced in high yield.

This facilitates producing a fluorinated polyether with a desired number-average molecular weight. Since a fluorine gas is not consumed for fluorination of a low molecular weight component and a high molecular weight component in the raw material polyether compound, the amount of fluorine gas used can be reduced, leading to cost reduction.

### BRIEF DESCRIPTION OF DRAWING

FIG. 1 is a ¹H-NMR spectrum of the fluorination raw material in Example 1.

FIG. 2 is a ¹H-NMR spectrum of the product of the step (4) in Example 1.

FIG. 3 is a ¹⁹F-NMR spectrum of the product of the step (4) in Example 1.

FIG. 4 is a GPC chart of the fluorination raw material in Example 1.

FIG. 5 is a GPC chart of the product of the step (4) in Example 1.

### DESCRIPTION OF EMBODIMENT

A method for producing the fluorinated polyether according to one embodiment of the present invention will be described in detail below.

The production method of the present embodiment is a method for producing a fluorinated polyether, characterized by including: a step (1) of performing an operation of adjusting a molecular weight distribution of a polymeric compound having the structural unit (R₁—O) to obtain the raw material compound represented by a formula (X); and after the step (1), a step (2) of introducing the raw material compound represented by the formula (X), an inert gas, a fluorine gas, and a solvent into a reactor to fluorinate the raw material compound.

R₄—O—(R₁—O)ₓ—R₅ (X)

wherein R₁ represents a divalent hydrocarbon group having 2 to 5 carbon atoms; R₁ in each structural unit represented by (R₁—O) may all be the same, or may be partially or totally different from each other; R₄ and R₅ each independently represent a protecting group for a hydroxyl group; x represents an average degree of polymerization and is a real number of 2.7 to 15.

### Step (1)

In the step (1) of the production method of the present embodiment, an operation of adjusting a molecular weight distribution of a polymeric compound having the structural unit (R₁—O) is performed to obtain the raw material compound represented by the formula (X).

### [Raw Material Compound Represented by the Formula (X)]

In the formula (X), R₁ in each structural unit independently represents a divalent hydrocarbon group having 2 to 5 carbon atoms. The hydrocarbon group may be a linear hydrocarbon group or a branched hydrocarbon group. R₁ in the formula (X) is preferably a hydrocarbon group having 2 to 4 carbon atoms, and examples thereof include —CH₂—CH₂—, —CH₂—CH₂—CH₂—, —CH₂—CH(CH₃)—, —CH(CH₃)—CH₂—, —CH₂—CH₂—CH₂—CH₂—, —CH(CH₃)—CH₂—CH₂—, —CH₂—CH(CH₃)—CH₂—, and —CH₂—CH₂—CH(CH₃)—. R₁ in the formula (X) is more preferably a linear hydrocarbon group, i.e., —CH₂—CH₂—, —CH₂—CH₂—CH₂—, —CH₂—CH₂—CH₂—CH₂—, and further preferably —CH₂—CH₂—CH₂—.

In the formula (X), R₁ in each structural unit represented by (R₁—O) may all be the same, or may be partially or totally different from each other. Namely, the raw material compound represented by the formula (X) may be a homopolymer (single polymer) in which R₁ in each structural unit represented by (R₁—O) is all the same, or may be a copolymer in which R₁ in each structural unit represented by (R₁—O) is at least partially different.

In a case of the raw material compound represented by the formula (X) being a copolymer, the number of types of structural units represented by (R₁—O) is not particularly limited. A sequence order of structural units is also not particularly limited, and may be a random sequence, a block sequence, and an alternating sequence, for example.

In the formula (X), x represents an average degree of polymerization and is a real number of 2.7 to 15. x is an average degree of polymerization, and therefore is not necessarily an integer. x is preferably a real number of 2.8 to 12, more preferably a real number of 2.9 to 10, and further preferably a real number of 3 to 8. In the case of the raw material compound represented by the formula (X) being a copolymer, x represents the total value of average degrees of polymerization for each type of structural unit.

In the case of the raw material compound represented by the formula (X) being a homopolymer, examples thereof include the compound represented by a formula (X-a) as the raw material compound.

R₄—O—(R₁ₐ—O)ₓₐ—R₅) (X-a)

wherein R₁ₐ represents a divalent hydrocarbon group having 2 to 5 carbon atoms, and the Rias, the number of which is xa, are all the same; R₄ and R₅ each independently represent a protecting group for a hydroxyl group; xa represents an average degree of polymerization and is a real number of 2.7 to 15.

In the case of the raw material compound represented by the formula (X) being a copolymer, examples thereof include the compound represented by a formula (X-b) or a formula (X-c) as the raw material compound.

R₄—O—(R_{1b}—O)_{xb}—(R_{1c}—O)_{xc}—R₅ (X-b)

wherein R_{1b} and R_{1c} each independently represent a divalent hydrocarbon group having 2 to 5 carbon atoms, and R_{1b} and R_{1c} have different structures; R₄ and R₅ each independently represent a protecting group for a hydroxyl group; xb and xc each represent an average degree of polymerization, and a sum of xb and xc is a real number of 2.7 to 15. A sequence order of the structural units represented by (R_{1b}—O) and (R_{1c}—O) is not limited.

R₄—O—(R_{1d}—O)_{xd}—(R₁ₑ—O)ₓₑ—(R_{1f}—O)_{xf}—R₅ (X-c)

wherein R_{1d}, R₁ₑ, and R_{1f} each independently represent a divalent hydrocarbon group having 2 to 5 carbon atoms, and R_{1d}, R₁ₑ, and R_{1f} are different structures from each other; R₄ and R₅ each independently represent a protecting group for a hydroxyl group; xd, xe, and xf each represent an average degree of polymerization, and the sum of xd, xe, and xf is a real number of 2.7 to 15; a sequence order of the structural units represented by (R_{1d}—O), (R₁ₑ—O) and (R_{1f}—O) is not particularly limited.

In the case of the raw material compound represented by the formula (X) being a copolymer, it is specifically preferably the compound represented by the formula (X-b), in which a combination of R_{1b} and R_{1c} is a combination of two selected from —CH₂—CH₂—, —CH₂—CH₂—CH₂—, and —CH₂—CH₂—CH₂—CH₂—.

In the formula (X), R₄ and R₅ each independently represent a protecting group for a hydroxyl group.

Examples of the protecting group for the hydroxyl group include an acyl group, an alkoxycarbonyl group, a silyl group, and an alkyl group that may have a substituent. R₄ and R; may be the same or different. R₄ and R₅ being the same as each other, facilitates synthesis and is preferred.

The acyl group is preferably represented as —(C═O)—R₈ wherein R₈ in the formula is a hydrogen atom or a hydrocarbon group having 1 to 8 carbon atoms, which may have a substituent. The hydrocarbon group more preferably has 1 to 3 carbon atoms.

In the case of R₈ being an alkyl group having 1 to 8 carbon atoms, which may have a substituent, the alkyl group may be linear or have a branch. Examples of the substituent include, for example, an alkoxy group, a fluoro group, a chloro group, and a bromo group.

When R₈ is an aryl group having 1 to 8 carbon atoms, which may have a substituent, examples of the substituent include, for example, an alkoxy group, a fluoro group, a chloro group, a bromo group, an acetoxy group, and a nitro group.

Specific examples of the acyl group include a formyl group, an acetyl group, an ethoxyacetyl group, a fluoroacetyl group, a difluoroacetyl group, a trifluoroacetyl group, a chloroacetyl group, a dichloroacetyl group, a trichloroacetyl group, a bromoacetyl group, a dibromoacetyl group, a tribromoacetyl group, a propionyl group, a 2-chloropropionyl group, a 3-chloropropionyl group, a pentafluoropropionyl group, a butyryl group, a 2-chlorobutyryl group, a 3-chlorobutyryl group, a 4-chlorobutyryl group, a 2-methylbutyryl group, a 2-ethylbutyryl group, a heptafluorobutyryl group, a valeryl group, a 2-methylvaleryl group, a 4-methylvaleryl group, a perfluorovaleryl group, a hexanoyl group, a perfluorohexanoyl group, a heptanoyl group, a perfluoroheptanoyl group, an octanoyl group, a perfluorooctanoyl group, a nonanoyl group, a perfluorononanoyl group, an isobutyryl group, an isovaleryl group, a pivaloyl group, a benzoyl group, an o-chlorobenzoyl group, a m-chlorobenzoyl group, a p-chlorobenzoyl group, an o-acetoxy benzoyl group, a m-acetoxy benzoyl group, a p-acetoxy benzoyl group, an o-methoxybenzoyl group, a m-methoxybenzoyl group, a p-methoxybenzoyl group, an o-nitrobenzoyl group, a m-nitrobenzoyl group, a p-nitrobenzoyl group, an o-fluorobenzoyl group, a m-fluorobenzoyl group, a p-fluorobenzoyl group, and a pentafluorobenzoyl group.

Specific examples of the alkoxycarbonyl group include a methoxycarbonyl group, an ethoxycarbonyl group, a 2,2,2-trichloroethoxycarbonyl group, and an allyloxycarbonyl group.

Specific examples of the silyl group include a trimethylsilyl group, a triethylsilyl group, a t-butyldimethylsilyl group, and a t-butyldiphenylsilyl group.

Examples of the alkyl group that may have a substituent include, for example, an alkyl group with a substituent selected from the group consisting of an alkoxy group, an aryl group, and a halogenated acyl group, and an alkyl group without a substituent. The number of carbon atoms of the alkyl group is not particularly limited, and an alkyl group having 1 to 8 carbon atoms is usually used.

Specific examples of the alkyl group having an alkoxy group include a methoxymethyl group, a methoxyethoxymethyl group, and an 1-ethoxyethyl group. The alkyl group having an alkoxy group may be a cyclic ether forming an acetal structure or ketal structure together with an oxygen atom derived from the hydroxyl group in the formula (X), and specific examples thereof include a 2-tetrahydropyranyl group.

Specific examples of the alkyl group having an aryl group include a benzyl group, a trityl group, an o-methoxybenzyl group, a m-methoxybenzyl group, and a p-methoxybenzyl group.

Specific examples of the alkyl group having a halogenated acyl group include —CH₂C(═O)F, —CH₂C(═O)Cl, —CH₂CH₂C(═O)F, —CH₂CH₂C(═O)Cl, —CH₂CH₂CH₂C(═O)F, and —CH₂CH₂CH₂C(═O)Cl.

Specific examples of the alkyl group without a substituent include a methyl group, an ethyl group, an n-propyl group, an i-propyl group, an n-butyl group, and a t-butyl group.

Among the above, R₄ and R₅ are each more preferably an acyl group, further preferably an acetyl group, a trifluoroacetyl group, a propionyl group, a pentafluoropropionyl group, a butyryl group, and a heptafluorobutyryl group, and particularly preferably an acetyl group or a trifluoroacetyl group.

### [Operation of Adjusting Molecular Weight Distribution]

In the step (1), an operation of adjusting a molecular weight distribution of a polymeric compound having the structural unit (R₁—O) (hereinafter sometimes referred to as a “compound to be adjusted”) is performed.

The compound to be adjusted has the same structural unit as that in the formula (X). Namely, the compound to be adjusted has structural units which are the same combination of (R₁—O) as that in the formula (X).

As the compound to be adjusted, both a commercially available product and a synthetic product can be used, and the compound usually has a wide molecular weight distribution, ranging from a low molecular weight component to a high molecular weight component. In the present invention, this molecular weight distribution is adjusted to obtain the raw material compound represented by the formula (X) described above.

For example, in a case in which the raw material compound represented by the formula (X-a) that is a homopolymer is obtained by the step (1), the compound to be adjusted is a polymeric compound having a structural unit represented by (R₁ₐ—O).

For example, in a case in which the raw material compound represented by the formula (X-b) that is a copolymer is obtained by the step (1), the compound to be adjusted is a polymeric compound having structural units represented by (R_{1b}—O) and (R_{1c}—O).

For example, in a case in which the raw material compound represented by the formula (X-c) that is a copolymer is obtained by the step (1), the compound to be adjusted is a polymeric compound having structural units represented by (R_{1d}—O), (R₁ₑ—O), and (R_{1f}—O).

Specific examples of the compound to be adjusted include the compound represented by the formula (A) or the formula (B). In the formula (A) and the formula (B), R; is the same as defined for the formula (X). That is, the compounds represented by the formula (A) and the formula (B) may be homopolymers or copolymers.

The compound represented by the formula (A) is a compound in which hydroxyl groups of a polyether compound are protected. The compound represented by the formula (B) is a compound in which hydroxyl groups of a polyether compound are not protected.

R₂—O—(R₁—O)ₚ—R₃ (A)

wherein R₁ is the same as defined for the formula (X); R₂ and R₃ each independently represent a protecting group for a hydroxyl group; p represents an average degree of polymerization and is a real number of 1 or greater.

HO—(R₁—O)_{q}—H (B)

wherein R₁ is the same as defined for the formula (X); q represents an average degree of polymerization and is a real number of 1 or greater.

In one embodiment, the step (1) includes the step (1A) of adjusting a molecular weight distribution of the compound represented by the formula (A). The compound represented by the formula (A) may be a commercially available product or a synthetic product.

In the case of the step (1) including the step (1A), the step (1) may have a step of protecting hydroxyl groups of a polyether compound in which hydroxyl groups are not protected (a compound in which hydrogen atoms are bonded in place of R₂ and R₃ in the formula (A)) to obtain the compound represented by the formula (A) before the step (1A). The polyether compound in which hydroxyl groups are not protected may be a commercially available product or a synthetic product. When synthesizing the polyether compound in which hydroxyl groups are not protected, it is preferably synthesized by sequential polymerization.

As the protecting groups represented by R₂ and R₃ in the formula (A), the protecting groups exemplified as R₄ and R₅ in the formula (X), can be used. R₂ and R₃ in the formula (A) may be the same as or different from R₄ and R₅ in the formula (X). In other words, protecting groups different from R₄ and R; in the formula (X) may be used as R₂ and R₃ in the formula (A), and a step of replacing the protecting groups may be carried out after the step (1A) to obtain the raw material compound represented by the formula (X). In this case, a protecting group suitable for adjusting a molecular weight distribution and a protecting group suitable for a fluorination reaction can be selected and used, respectively. In the case of R₂ and R₃ in the formula (A) being the same as R₄ and R₅ in the formula (X), the compound obtained in the step (1A) can be used as a raw material compound for the fluorination reaction, which is preferred.

p representing an average degree of polymerization in the formula (A) is a real number of 1 or greater, and it may be a real number of 1.5 to 30, or a real number of 2 to 20.

In one embodiment, the step (1) includes the step (1B) of adjusting a molecular weight distribution of the compound represented by the formula (B). The compound represented by the formula (B) may be a commercially available product or a synthetic product. When synthesizing the compound represented by the formula (B), it is preferably synthesized by sequential polymerization.

q representing an average degree of polymerization in the formula (B) is a real number of 1 or greater, and it may be a real number of 1.5 to 30, or a real number of 2 to 20.

The step (1), in the case of including the step (1B), preferably includes the step (1C) of protecting a hydroxyl group of the compound obtained by the step (1B) after the step (1B).

As protecting groups used to protect hydroxyl groups in the step (1C), the protecting groups exemplified as R₄ and R; in the formula (X) can be used, and they may be the same as or different from R₄ and R₅ in the formula (X). In the case of the protecting groups used to protect hydroxyl groups in the step (1C) being the same as R₄ and R₅ in the formula (X), the compound obtained in the step (1C) can be used as a raw material compound for the fluorination reaction, which is preferred.

The step (1), in the case of including the step (1B) and the step (1C), may include, after the step (1C), the step (1D) of adjusting a molecular weight distribution of the compound obtained by the step (1C). By performing the step of adjusting a molecular weight distribution two times, it is possible to adjust the molecular weight distribution at a higher level.

In the case of having protected hydroxyl groups in the step (1C) with protecting groups different from R₄ and R₅ in the formula (X), a step of replacing the protecting group may be carried out after the step (1D) to obtain the raw material compound represented by the formula (X). In this case, a protecting group suitable for adjusting a molecular weight distribution and a protecting group suitable for a fluorination reaction may be selected and used, respectively. In the case of having protected hydroxyl groups in the step (1C) with the same protecting groups as R₄ and R₅ in the formula (X), the compound obtained in the step (1D) can be used as a raw material compound for the fluorination reaction, which is preferred.

When synthesizing a polyether compound in which hydroxyl groups are not protected (a compound in which hydrogen atoms are bonded in place of R₂ and R₃ in the formula (A), or the compound represented by the formula (B)) by sequential polymerization, a conventionally known method can be used as the synthetic method. For example, a synthesis method by a polymerization reaction of diol, and a synthesis method by a ring-opening polymerization of cyclic ether, can be employed.

The polyether compound in which hydroxyl groups are not protected (the compound in which hydrogen atoms are bonded in place of R₂ and R₃ in the formula (A), or the compound represented by the formula (B)) can also be synthesized by a nucleophilic substitution reaction between compounds having a polyether chain (or a monomer unit that will constitute a polyether chain).

For example, a method can be used of reacting a compound having a leaving group at one end of a polyether chain (or a monomer unit that will constitute a polyether chain) and a protected hydroxyl group at the other end (hereinafter sometimes referred to as a “compound having a leaving group at one end”), and a compound having a hydroxyl group at one end of a polyether chain (or a monomer unit that will constitute a polyether chain) and a protected hydroxyl group at the other end (hereinafter sometimes referred to as a “protected diol compound”), to elongate the polyether chain. The compound having a leaving group at one end can be reacted with the protected diol compound at a molar ratio of approximately 1:1, to elongate the polyether chain at one end of the protected diol compound.

The above compound having a leaving group at one end and a compound having hydroxyl groups at both ends of a polyether chain (or a monomer unit that will constitute a polyether chain) (hereinafter sometimes referred to as a “diol compound”) may be reacted. The compound having a leaving group at one end can be reacted with the diol compound at a molar ratio of approximately 2:1 to elongate the polyether chain at both ends of the diol compound.

The above protected diol compound and a compound having leaving groups at both ends of a polyether chain (or a monomer unit that will constitute a polyether chain) (hereinafter sometimes referred to as a “compound having leaving groups at both ends”), may be reacted. The protected diol compound can be reacted with the compound having leaving groups at both ends at a molar ratio of approximately 2:1, to elongate the polyether chain at both ends of the compound having leaving groups at both ends.

In the case of having a step of protecting hydroxyl groups of a polyether compound in which hydroxyl groups are not protected (a compound in which hydrogen atoms are bonded in place of R₂ and R₃ in the formula (A), or the compound represented by the formula (B)), a reacting agent used for the protection can be appropriately selected according to the type of protecting group. For example, in a case in which the protecting group is an acyl group and is represented by —(C═O)—R₈ wherein in the formula, R₉ is a hydrogen atom or a hydrocarbon group having 1 to 8 carbon atoms, which may have a substituent, an acylating agent that is an acid halide such as R₈—(C═O)Cl or R₉—(C═O)F, or an acid anhydride such as R₈—(C═O)—O—(C═O)—R₈, can be used.

The acylating agent is specifically preferably acid halides such as CH₃(C═O)F, CH₃(C═O)Cl, CF₃(C═O)Cl, CCl₃ (C═O)Cl, CH₃CH₂(C═O)F, CH₃CH₂(C═O)Cl, CF₃CF₂(C═O)F, CF₃CF₂(C═O)Cl, CH₃CH₂CH₂(C═O)F, CH₃CH₂CH₂ (C═O)Cl, CF₃CF₂CF₂(C═O)F, CF₃CF₂CF₂ (C═O)Cl, and CF₃CF₂CF₂CF₂CF₂ (C═O)F; and acid anhydrides such as CH₃(C═O)—O—(C═O)CH₃, CF₃(C═O)—O—(C═O)CF₃, CCl₃(C═O)—O—(C═O)CCl₃, CH₃CH₂ (C═O)—O—(C═O) CH₂CH₃, CF₃CF₂ (C═O)—O—(C═O) CF₂CF₃, CH₃CH₂CH₂ (C═O)—O—(C═O) CH₂CH₂CH₃, and CF₃CF₂CF₂ (C═O)—O—(C═O) CF₂CF₂CF₃.

The operation of adjusting a molecular weight distribution in the step (1) is preferably at least one operation selected from an operation of adjusting a molecular weight distribution of a polymeric compound having the same structural unit as that in the formula (X) so as to become narrower, and an operation of mixing two or more types of monodisperse polymers of a polymeric compound having the same structural unit as that in the formula (X) to adjust the molecular weight distribution.

In the case of carrying out a step of adjusting a molecular weight distribution two or more times in the step (1), the operations of adjusting a molecular weight distribution in each step may be the same or different. For example, in the case of the step (1) including the steps (1B), (1C), and (1D) above, the operations of adjusting a molecular weight distribution in the step (1B) and the step (1D) may be the same or different.

In the case of performing an operation of adjusting a molecular weight distribution of a polymeric compound having the same structural unit as that in the formula (X) so as to become narrower in the step (1), a compound to be adjusted is a compound having a wide molecular weight distribution. Mw/Mn of the compound to be adjusted (Mw is a weight-average molecular weight and Mn is a number-average molecular weight) is not particularly limited as long as Mw/Mn is a greater value than Mw/Mn of the raw material compound represented by the formula (X) to be obtained in the step (1). Normally, Mw/Mn of the compound to be adjusted is greater than 1.30, but a molecular weight distribution of a compound to be adjusted, Mw/Mn of which is 1.30 or smaller, may be adjusted so as to become further narrower.

The operation of adjusting a molecular weight distribution so as to become narrower (hereinafter sometimes referred to as an “adjustment method 1”) is not particularly limited, but includes chromatography, distillation, extraction, crystallization, and filtration, for example.

In the case of using chromatography as the adjustment method 1, silica gel column chromatography is preferred. Examples thereof include a method of performing fractionation by using a column packed with silica gel having a particle size (diameter) of 30 to 70 μm in a 10 to 100-fold amount (mass ratio) of the compound to be adjusted. In the silica gel column chromatography, examples of solvents to dissolve or disperse the compound to be adjusted include a single or mixed solvent selected from the group consisting of hexane, ethyl acetate, toluene, methylene chloride, methanol, ethanol, and isopropyl alcohol.

In the case of performing distillation as the adjustment method 1, fractional distillation under normal pressure or reduced pressure enables removal of a component contained in the compound to be adjusted and having a degree of polymerization that is unnecessary, or selective acquisition of a component contained in the compound to be adjusted and having a degree of polymerization that is necessary. An appropriate reflux ratio may be set in order to increase separation accuracy of compounds, or a filler may be used for rectification distillation.

In the case of performing extraction as the adjustment method 1, for example, allowing the compound to be adjusted to dissolve in water or an organic solvent followed by use of a solvent that is immiscible with it, enables removal of a component having a degree of polymerization that is unnecessary, or acquisition of a component having a degree of polymerization that is necessary. A type of solvent used for extraction, a mixing ratio of the solvents, the number of extractions, and the like, can be adjusted according to the type of compound to be adjusted and a molecular weight of a component to be extracted.

In the case of performing crystallization as the adjustment method 1, for example, by a method of cooling a solution obtained by dissolving the compound to be adjusted in water or an organic solvent, or by a method of adding a poor solvent to the solution obtained by dissolving the compound to be adjusted in water or an organic solvent, the compound to be adjusted can partially precipitate as solid and can be separated. A type of solvent used for crystallization, a mixing ratio of the solvents, a crystallization temperature, and the like, can be adjusted according to the type of compound to be adjusted and a molecular weight of a component to be precipitated.

In the case of the raw material compound represented by the formula (X) obtained by the step (1) being a homopolymer (for example, the compound represented by the formula (X-a)), as the adjustment method 1, a method of mixing the compound to be adjusted with a monodisperse polymer having the same structural unit as that in the compound and a molecular weight close to the peak of a molecular weight distribution of the compound to be adjusted, can also be used. By mixing the monodisperse polymer, the amount of the central molecular weight component in a molecular weight distribution becomes relatively larger than those of low molecular weight component and high molecular weight component, resulting in a narrower molecular weight distribution.

The monodisperse polymer refers to a polymer substantially composed of a compound with a single degree of polymerization and usually has no molecular weight distribution (Mw/Mn=1). However, it is industrially difficult to use a completely monodisperse polymer, and therefore Mw/Mn of the monodisperse polymer can be 1.00 or more and smaller than 1.02, and Mw/Mn of the monodisperse polymer is preferably 1.00 or greater and 1.01 or smaller. For example, in the case of the single degree of polymerization being an integer represented by m, a proportion of a compound with a degree of polymerization other than m, contained in a monodisperse polymer can be 3% or less in terms of peak area ratio based on GPC analysis.

The monodisperse polymer may be obtained by performing an operation of adjusting a molecular weight distribution so as to become narrower, or the monodisperse polymer may be synthesized by a nucleophilic substitution reaction between the compounds having a polyether chain (or a monomer unit that will constitute a polyether chain), as described above.

A mixing ratio of a compound to be adjusted and a monodisperse polymer, and a degree of polymerization of a monodisperse polymer to be mixed, can be adjusted according to a molecular weight distribution of the compound to be adjusted. For example, in a case in which the compound to be adjusted is the compound represented by the formula (A): R₂—O—(R₁—O)ₚ—R₃ wherein the definitions of R₁, R₂, R₃, and p are as described above, having a molecular weight distribution, and a degree of polymerization of a compound contained in the highest proportion in the compound to be adjusted is 5, mixing the monodisperse polymer represented by R₂—O—(R₁—O)₅—R₃ wherein R₁, R₂, and R₃ are the same as defined for the formula (A) with the compound to be adjusted, enables adjustment of molecular weight distribution of the compound to be adjusted so as to become narrower.

In the step (1), when performing an operation of adjusting a molecular weight distribution of a polymeric compound having the same structural unit as that in the formula (X) so as to become narrower, a difference in Mw/Mn before and after the adjustment (=(Mw/Mn of the compound to be adjusted)−(Mw/Mn of the compound after adjustment)) is greater than 0, and it may be 0.05 or more, may be 0.10 or more, may be 0.15 or more, or may be 0.20 or more.

In the step (1), an operation of mixing two or more types of monodisperse polymers of a polymeric compound having the same structural unit as that in the formula (X) to adjust the molecular weight distribution (hereinafter sometimes referred to as an “adjustment method 2”) may be performed. The monodisperse polymer is as described above. In the adjustment method 2, the number of the type of monodisperse polymer to be mixed may be two types, three types, or four or more types. By carrying out the adjustment method 2, it is possible to obtain the raw material compound represented by the formula (X) that is free of a low molecular weight component and a high molecular weight component, which are undesirable. Carrying out the adjustment method 2 enables adjusting a molecular weight distribution to have a discontinuous degree of polymerization.

A mixing ratio of a monodisperse polymer and a degree of polymerization of the monodisperse polymer to be mixed in the adjustment method 2 may be selected so that a molecular weight distribution after an operation of adjusting it falls within a desired range. Examples of the method for adjusting a molecular weight distribution to have a discontinuous degree of polymerization include a method of mixing the monodisperse polymers represented by R₂—O—(R₁—O)₃—R₃ and R₂—O—(R₁—O)₇—R₃ wherein the definitions of R₁, R₂, and R₃ are as described above.

Mw/Mn representing a molecular weight distribution of the raw material compound represented by the formula (X) obtained in the step (1) is preferably 1.30 or smaller, more preferably 1.20 or smaller, further preferably 1.10 or smaller, and particularly preferably 1.05 or smaller.

In the raw material compound represented by the formula (X) obtained in the step (1), a number-average molecular weight of the structure represented by —O—(R₁—O)ₓ— (structure excluding R₄ and R₅ in the formula (X)), is preferably 130 or more and 1,300 or less and more preferably 170 or more and 870 or less.

The total proportion of a compound represented by the formula (X-1) contained in the raw material compound represented by the formula (X) obtained in the step (1) is preferably 5% or less in terms of peak area ratio based on GPC analysis.

R₄—O—(R₁—O)ᵣ—R₅ (X-1)

wherein R₁, R₄, and R₅ are the same as defined for the formula (X); and r represents 1 or 2.

The compound represented by the formula (X-1) has the same R₁, R₄, and R₅ as those of the formula (X) and r representing a degree of polymerization of 1 or 2, representing a low molecular weight component. In the step (1), the operation of adjusting a molecular weight distribution preferably removes a low molecular weight component. Reduction of low molecular weight component in the step (1) facilitates obtaining a fluorinated polyether with a number-average molecular weight as theoretically calculated from a number-average molecular weight of a raw material compound in high yield, in the step (2).

The total proportion of a compound represented by the formula (X-1) contained in the raw material compound is more preferably 3% or less in terms of peak area ratio based on GPC analysis, further preferably 2% or less, and particularly preferably 1% or less.

A method for calculating a peak area ratio of the compound represented by the formula (X-1) based on GPC analysis is determined by the method described in Examples.

In particular, in a case in which the raw material compound represented by the formula (X) obtained in the step (1) is a homopolymer (for example, the compound represented by the formula (X-a)) in which R₁ in each structural unit in the formula (X) is all the same, the total proportion of a compound represented by the formula (X-1) in the raw material compound falling within the above range facilitates obtaining a fluorinated polyether in high yield.

The total proportion of a compound represented by the formula (X-2) contained in the raw material compound represented by the formula (X) obtained in the step (1) is preferably 15% or less in terms of peak area ratio based on GPC analysis.

R₄—O—(R₁—O)ₛ—R₅ (X-2)

wherein R₁, R₄, and R₅ are the same as defined for the formula (X). s is an integer and satisfies s≥(average degree of polymerization x of the formula (X)+4).

The compound represented by the formula (X-2) has the same R₁, R₄, and R₅ as those of the formula (X) and s representing a degree of polymerization of an integer value satisfying s≥(average degree of polymerization x of the formula (X)+4), representing a high molecular weight component. In the step (1), the operation of adjusting a molecular weight distribution preferably removes a high molecular weight component. Reduction of high molecular weight component in the step (1) facilitates obtaining a fluorinated polyether with a number-average molecular weight as theoretically calculated from a number-average molecular weight of a raw material compound in high yield in the step (2).

The total proportion of a compound represented by the formula (X-2) in the raw material compound is more preferably 10% or less, further preferably 5% or less, and particularly preferably 1% or less, in terms of peak area ratio based on GPC analysis.

A method for calculating a peak area ratio of the compound represented by the formula (X-2) based on GPC analysis is determined by the method described in Examples.

In particular, in a case in which the raw material compound represented by the formula (X) obtained in the step (1) is a homopolymer (for example, the compound represented by the formula (X-a)) in which R₁ in each structural unit of the formula (X) is all the same, the total proportion of a compound represented by the formula (X-2) contained in the raw material compound falling within the above range facilitates obtaining a fluorinated polyether in high yield.

It is preferred that the total proportion of a compound represented by the formula (X-1) contained in the raw material compound represented by the formula (X) obtained in the step (1) is 5% or less in terms of peak area ratio based on GPC analysis, and the total proportion of a compound represented by the formula (X-2) is 15% or less in terms of peak area ratio based on GPC analysis. In this case, both a low molecular weight component and a high molecular weight component are reduced in the raw material compound, resulting in further facilitating obtaining, in the step (2), a fluorinated polyether with a number-average molecular weight as theoretically calculated from a number-average molecular weight of the raw material compound in high yield.

In particular, in a case in which the raw material compound represented by the formula (X) obtained in the step (1) is a homopolymer (for example, the compound represented by the formula (X-a)) in which R₁ in each structural unit in the formula (X) is all the same, the total proportion of a compound represented by the formula (X-1) and the total proportion of a compound represented by the formula (X-2) in the raw material compound falling within the above ranges facilitate obtaining a fluorinated polyether in high yield.

### Step (2)

In the production method of the present embodiment, in the step (2), the raw material compound represented by the formula (X), an inert gas, a fluorine gas, and a solvent are introduced into a reactor to fluorinate the raw material compound.

The equivalent of fluorine gas introduced into a reactor is preferably 1.0 to 5.0 equivalents, and more preferably 1.1 to 3.0 equivalents relative to the number of moles of hydrogen atoms contained in a raw material compound. The equivalent of fluorine gas being 1.0 equivalent or more relative to the number of moles of hydrogen atoms contained in a raw material compound, facilitates sufficient proceeding of fluorination reaction. The equivalent of fluorine gas being 5.0 equivalents or less relative to the number of moles of hydrogen atoms contained in a raw material compound, can prevent unconsumed fluorine gas from being wasted.

A concentration of a fluorine gas to be distributed in a reactor is preferably 1 to 30% by volume, more preferably 10 to 20% by volume, based on the total volume of distribution gas (fluorine gas+inert gas). The fluorine gas concentration of 1% by volume or more can prevent the reaction time from becoming long due to a reduced reaction rate. The fluorine gas concentration being 30% by volume or less can prevent reaction runaway and generation of side reaction. Pressure in a reactor upon introduction of fluorine gas is preferably 0.08 to 0.12 MPa, more preferably normal pressure (0.1 MPa) to 0.115 MPa. The pressure of 0.12 MPa or lower can prevent the reaction runaway and generation of side reaction.

An inert gas is distributed into a reactor so that the fluorine gas concentration falls within the above range. The inert gas and fluorine gas may be introduced in separate systems, or a mixture gas of inert gas and fluorine gas, in which the fluorine gas has been preliminarily diluted with the inert gas, may be introduced into a reactor. As the inert gas, a nitrogen gas, a helium gas, and an argon gas are preferred because of their easy availability and handleability.

A solvent used in a fluorination reaction is not particularly limited, but a solvent with high solubility for a raw material compound and a product, which is the fluorinated polyether, is preferred, and a solvent that does not react with the raw material compound, the product, and a fluorine gas, are more preferred. Specifically, a solvent that is fully halogen-substituted and free of carbon-carbon unsaturated bonds, is preferred. The solvent that is fully halogen-substituted and free of carbon-carbon unsaturated bonds, is free of a C—H bond and a carbon-carbon unsaturated bond, and therefore, a C—H bond or a carbon-carbon unsaturated bond in the solvent does not react with the fluorine gas, and an increase in the amount of fluorine gas used and a temperature rise due to reaction heat can be prevented. A decomposition reaction of the raw material compound by hydrogen fluoride, which is generated in the case of reaction between the C—H bond and the fluorine gas, also does not occur, which is preferred.

Examples of the solvent used in the fluorination reaction include a perhalogenated alkane, a perhalogenated polyether, a perhalogenated carboxylic acid or an anhydride thereof. The solvent may be used singly or in combinations with two or more types.

The perhalogenated alkane having 2 to 8 carbon atoms is preferred. The perhalogenated alkane containing a fluorine atom and a chlorine atom is more preferable from the viewpoint of solubility for a raw material compound, and includes, for example, dichlorotetrafluoroethane, trichlorotrifluoroethane, dichlorohexafluoropropane, and tetrachlorohexafluorobutane.

Examples of commercially available perhalogenated polyethers include, for example, DEMNUM (registered trademark) by DAIKIN INDUSTRIES, LTD., FLUORINERT (registered trademark) by 3M Japan Limited, GALDEN (registered trademark) by Solvay Specialty Polymers Japan K.K., KRYTOX (registered trademark) by The Chemours Company.

Examples of the perhalogenated carboxylic acid or anhydride thereof include trifluoroacetic acid and trifluoroacetic anhydride.

The solvent used in the fluorination reaction is preferably introduced into a reactor before introducing a raw material compound. It is preferable that an inert gas and a fluorine gas are distributed in a reactor before introducing the raw material to saturate a solvent with the fluorine gas.

A preferred method for introducing the raw material compound, is a method of preparing the raw material solution obtained by dissolving the raw material compound in a solvent, and supplying the raw material solution into a reactor while distributing the inert gas and the fluorine gas in the reactor. As the solvent dissolving the raw material compound, solvents exemplified for the fluorination reaction can be used and is preferably the same as the solvent used in the fluorination reaction.

A concentration of the raw material compound in the reactor may be adjusted according to its solubility in the solvent, and it is preferably 0 to 3.0 mol/L and more preferably 0 to 1.5 mol/L. A supply rate of the raw material solution into the reactor may be adjusted so that the equivalent of the fluorine gas relative to the amount of raw material compound falls within the above range, depending on a concentration and a flow rate of the fluorine gas to be distributed.

In the step (2), a temperature in a reactor upon fluorine gas introduction is preferably −30 to 60° C. and more preferably −20 to 30° C.

In one embodiment, a temperature in a reactor upon the fluorine gas introduction is preferably 20 to 60° C. and more preferably 20 to 30° C. The temperature in the reactor is preferably higher than the boiling point of hydrogen fluoride (20° C.) in order to efficiently remove hydrogen fluoride that was produced as a by-product. In the case of the temperature being 20° C. or higher, hydrogen fluoride does not remain and a decomposition reaction of a raw material is less likely to occur, which is preferred. In the case of the temperature being 60° C. or lower, reaction runaway and generation of side reaction can be prevented, which is preferred.

In another embodiment, a temperature in a reactor upon fluorine gas introduction may be −30 to 20° C. or −20 to 0° C. In this case, it is preferable that in order to efficiently remove hydrogen fluoride that has been produced as a by-product, a dilution ratio of a nitrogen gas to a fluorine gas is increased or a hydrogen fluoride scavenger is used. Examples of the hydrogen fluoride scavenger include alkali metal fluorides such as sodium fluoride and potassium fluoride, and organic bases such as trialkylamine.

As a reactor in the fluorination reaction, a pressure-resistant reactor is preferably used, and an autoclave is usually used. A material of the reactor is not particularly limited, but a metal vessel made of stainless steel or nickel, or a vessel coated with a fluororesin is preferred because they are less reactive with a fluorine gas.

In the fluorination reaction, both of a distribution type reactor and a batch type reactor can be employed.

In the case of the distribution type reactor, a flow rate of a raw material solution supplied to the reactor is not particularly limited, but is adjusted according to the equivalent of fluorine gas relative to the amount of raw material compound, a size of the reactor, and pressure in the reactor, for example. The flow rate of the raw material solution supplied to the reactor is preferably 0.5 to 100 mmol/min and more preferably 2 to 30 mmol/min, based on the number of moles of hydrogen atoms contained in the raw material compound.

In the case of the batch type reactor, pressure-controlled fluorine gas may be introduced from an entrance of the reactor as much as is consumed in the reaction.

The step (2) yields a fluorinated polyether in which hydrogen atoms bonded to carbon atoms, contained in the raw material compound represented by the formula (X), have been replaced with fluorine atoms. The step (2) can produce, for example, the compound represented by formula (Xf).

Rf₄—O—(Rf₁—O)ₓ—Rf₅ (Xf)

wherein Rf₁ represents a divalent perfluorohydrocarbon group in which all hydrogen atoms of R₁ in the formula (X) were substituted with fluorine atoms; Rf₄ represents a group in which all hydrogen atoms of R₄ in the formula (X) were substituted with fluorine atoms; Rf₅ represents a group in which all hydrogen atoms of R₅ in the formula (X) were substituted with fluorine atoms; and x is the same as defined for the formula (X).

### Step (3)

In the production method of the present embodiment, following the step (2), the step (3) may be carried out in which a perhalogenated unsaturated hydrocarbon compound is introduced into a reactor while distributing an inert gas and a fluorine gas in the reactor.

In a later stage of the fluorination reaction in the step (2), a reaction rate of the fluorination reaction may be reduced. Therefore, following the step (2), the step (3) of introducing a perhalogenated unsaturated hydrocarbon compound into a reactor while distributing an inert gas and a fluorine gas, is preferably included. Introducing the perhalogenated unsaturated hydrocarbon compound allows an unsaturated bond in the perhalogenated unsaturated hydrocarbon compound to react with the fluorine gas to generate fluorine radicals. The generated fluorine radicals react with the raw material compound and fluorination proceeds, thereby enabling acceleration of the fluorination reaction by carrying out the step (3).

As the inert gas used in the step (3), the gas exemplified in the step (2) can be used. Flow rates of the inert gas and fluorine gas in the step (3) are preferably adjusted so that a concentration of the fluorine gas distributed in the reactor falls within the range exemplified in the step (2).

Examples of the perhalogenated unsaturated hydrocarbon compound include hexafluorobenzene, hexachlorobenzene, chloropentafluorobenzene, trichlorotrifluorobenzene, decafluorobiphenyl, octafluoronaphthalene, tetrachloroethylene, trichlorofluoroethylene, dichlorodifluoroethylene, trichlorotrifluoropropene, and dichlorotetrafluoropropene, with hexafluorobenzene being particularly preferred because of easy availability and handleability among them.

By using the perhalogenated unsaturated hydrocarbon compound in the step (3), the fluorine gas is not consumed for fluorination of C—H bonds in the unsaturated hydrocarbon compound, as is the case of using an unsaturated hydrocarbon compound with C—H bonds such as benzene, and the amount of fluorine gas used does not increase, which is preferred.

A method for introducing the perhalogenated unsaturated hydrocarbon compound in the step (3) is preferably a method of dissolving the perhalogenated unsaturated hydrocarbon compound in a solvent and introducing into distribution a fixed amount of the compound into a reactor. The amount of perhalogenated unsaturated hydrocarbon compound distributed is preferably 1/50 to 1/5 mol per mol, more preferably 1/30 to 1/10 mol per mol in terms of the number of moles of unsaturated bonds in the perhalogenated unsaturated hydrocarbon compound, relative to the amount of fluorine gas distributed. When the amount of perhalogenated unsaturated hydrocarbon compound distributed is 1/50 mol per mol or more, proceeding of the fluorination reaction is not delayed, thereby making it possible to prevent a reaction time from prolonging. When the amount of perhalogenated unsaturated hydrocarbon compound distributed is 1/5 mol per mol or less, the reaction runaway and generation of side reaction can be prevented.

Pressure in a reactor upon introduction of the perhalogenated unsaturated hydrocarbon compound is preferably 0.08 to 0.12 MPa, more preferably normal pressure (0.1 MPa) to 0.115 MPa. A temperature in the reactor upon introduction of the perhalogenated unsaturated hydrocarbon compound is preferably −30 to 60° C. and more preferably −20 to 30° C.

The same solvent as in the step (2) is preferably used as a solvent for the step (3). In the case of using a solvent different from that of the step (2), the solvent exemplified in the step (2) can be used.

In a case in which the perhalogenated unsaturated hydrocarbon compound is dissolved in a solvent and supplied to a reactor as a solution, a concentration of the perhalogenated unsaturated hydrocarbon compound in the supplying solution can be adjusted according to its solubility in the solvent, and the concentration is preferably 0.01 to 100 mol/L and more preferably 0.1 to 10 mol/L based on the number of moles of unsaturated bonds in the perhalogenated unsaturated hydrocarbon compound.

### Step (4)

In the production method of the present embodiment, following the step (2) or the step (3), the step (4) may be carried out in which a fluorinated polyether is reacted with an alcohol having 1 to 3 carbon atoms.

The fluorinated polyethers generated in the step (2) and/or the step (3) may readily react with moisture in the atmosphere to form carboxylic acid compounds. Therefore, considering handleability in the subsequent step, the step (4) is preferably carried out. In particular, in a case in which R₄ and R₅ are acyl groups in the raw material compound represented by the formula (X), it is preferable to perform the step (4).

Examples of the alcohol having 1 to 3 carbon atoms include methanol, ethanol, and n-propanol. Among them, methanol is preferred.

A reaction temperature in the step (4) is preferably −30 to 60° C. and more preferably −20 to 30° C. Reaction pressure is preferably 0.08 to 0.12 MPa and more preferably normal pressure (0.1 MPa) to 0.115 MPa.

The amount of alcohol introduced is preferably 2 to 10 equivalents and more preferably 3 to 5 equivalents, relative to the number of moles of reaction ends (theoretical amount based on the number of moles of a raw material compound) contained in the fluorinated polyether generated in the step (2) and/or the step (3).

In the case of R₄ and R₅ being acyl groups in the raw material compound represented by the formula (X), among carbon atoms contained in the structure represented by —O—(R₁—O)ₓ— (structure excluding R₄ and R₅ in the formula (X)), the outermost carbon atom converts to a carbonyl carbon atom and acyl groups (Rf₄ and Rf₅ in formula (Xf)) perfluorinated by the fluorination reaction are eliminated to generate acid fluoride. The acid fluoride reacts with an alcohol to generate a carboxylic acid ester.

For the case where the raw material compound represented by the formula (X) is a homopolymer, R₁ is —CH₂—CH₂—, and R₄ and R₅ are acetyl groups, reactions of the step (2) to the step (4) are shown in formula (i). The following formula represents a reaction when methanol is used in the step (4).

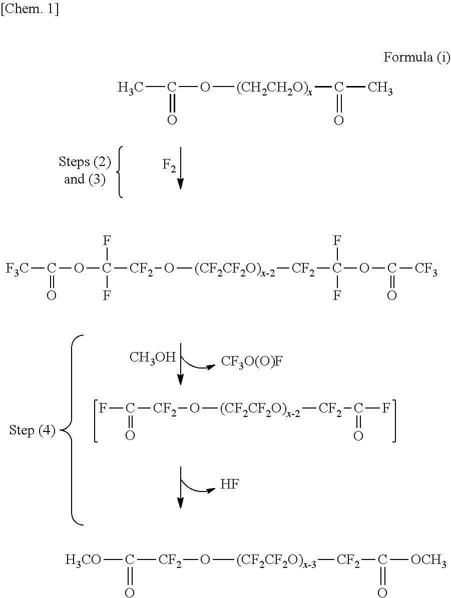

Carrying out the step (4), in which the fluorinated polyethers generated in the step (2) and/or the step (3) are reacted with an alcohol having 1 to 3 carbon atoms, can, for example, produce the compound represented by the formula (Y).

R₆O—(C═O)—Rf₂—O—(Rf₁—O)_{y}—Rf₃—(C═O)—OR₇ (Y)

wherein Rf₁ represents a divalent perfluorohydrocarbon group having 2 to 5 carbon atoms; Rf₁ in each structural unit represented by (Rf₁—O) may all be the same, or may be partially or totally different from each other; Rf₂ and Rf₃ each independently represent a perfluorohydrocarbon group having 1 to 4 carbon atoms and are determined according to a structure of a structural unit arranged at an end in the formula (X); R₆ and R₇ each independently represent an alkyl group having 1 to 3 carbon atoms; and y represents an average degree of polymerization and is a real number of 0.7 to 13.

In the formula (Y), the structure represented by —O—(Rf₁—O)_{y}—corresponds to a structure in which all hydrogen atoms of R₁ in a structure in which structural units arranged at both ends of the structure represented by —O—(R₁—O)ₓ—in the formula (X) are excluded, are replaced with fluorine atoms.

Therefore, in the formula (Y), a structure of Rf₁ in each structural unit represented by (Rf₁—O) and a sequence order of each structural unit represented by (Rf₁—O), are determined according to the structure in which structural units arranged at both ends of the structure represented by —O—(R₁—O)ₓ—in the formula (X) are excluded.

In the formula (Y), the structures represented by —(C═O)—Rf₂—and —Rf₃—(C═O)—are each derived from a structural unit arranged at each end of the structure represented by —O—(R₁—O)ₓ—in the formula (X). Therefore, in the formula (Y), structures of the perfluorohydrocarbon groups represented by Rf₂ and Rf₃ are determined according to a structure of a structural unit arranged at an end in the formula (X). The number of carbon atoms contained in each of perfluorohydrocarbon groups represented by Rf₂ and Rf₃ is one less than the number of carbon atoms contained in a structural unit arranged at each end of the structure represented by —O—(R₁—O)ₓ—in the formula (X).

In the formula (Y), the structures represented by R₆O— and —OR₇ are derived from an alcohol used in the step (4).

A case in which, in the raw material compound represented by the formula (X), a structural unit arranged at each end of the structure represented by —O—(R—O)ₓ— has a branched structure, will be described. In this case, hydroxyl groups protected by the protecting groups represented by R₄ and R₅ in the raw material compound represented by the formula (X) are not necessarily primary hydroxyl groups (for example, hydroxyl group of —CH₂OH), and can be secondary hydroxyl groups (for example, hydroxyl group of —CH(CH₃)OH) or tertiary hydroxyl groups (for example, hydroxyl group of —C(CH₃)₂OH). In a case in which the step (4) is carried out after fluorination of such raw material compounds, a structure derived from the primary hydroxyl group converts to a carboxylic acid ester (for example, —(C═O)OCH₃) as described above, while a structure derived from the secondary hydroxyl group is thought to convert to a ketone (for example, —(C═O)CF₃) by elimination of fluoride.

A theoretical value of number-average molecular weight of a product of the step (4) is preferably 350 or more and 4,000 or less and more preferably 500 or more and 2,600 or less.

The measured value/theoretical value of the number-average molecular weight of the product of the step (4) is preferably 0.9 or more and 1.1 or less and more preferably 0.95 or more and 1.05 or less.

The product of the step (2), the step (3) or the step (4) can be isolated as a residue after a solvent is distilled off. Before the solvent is distilled off, it is preferable to wash the product with alkaline water in order to remove a by-product such as hydrogen fluoride generated in the fluorination reaction. The alkaline water is not particularly limited, but due to easy availability and handleability, sodium carbonate water or sodium hydrogen carbonate water is preferable. After having washed with alkaline water followed by having collected a solvent layer from the separated two layers, in order to completely remove water and hydrogen fluoride, a hydrogen fluoride scavenger and a drying agent are preferably added and stirred.

Examples of the hydrogen fluoride scavenger include alkali metal fluorides such as sodium fluoride and potassium fluoride, and an organic base such as trialkylamine. The hydrogen fluoride scavenger is preferably solid alkali metal fluoride because of easy separation thereof, and particularly preferably sodium fluoride.

As the drying agent, sodium sulfate or magnesium sulfate is preferred.

After filtering off the solid, the product of the step (2), the step (3) or the step (4) can be isolated by distilling off a solvent, and the recovered solvent can be easily reused, enabling reduction of loss of expensive solvent such as a fully halogen-substituted compound.

### Step (5)

In the production method of the present embodiment, following the step (4), the step (5) of reducing esters at both ends of the compound obtained by the step (4) may be carried out. In a case in which the compound represented by the formula (Y) is obtained by the step (4), the compound represented by the formula (Z) can be produced by carrying out the step (5).

HO—CH₂—Rf₂—O—(Rf₁—O)_{y}—Rf₃—CH₂—OH (Z)

wherein Rf₁, Rf₂, Rf₃, and y are the same as defined for the formula (Y).

In the step (5), a known method for reducing the ester can be used. For example, a method of mixing the compound obtained by the step (4) with a reducing agent in a solvent, can be employed.

A solvent used in the step (5) is preferably an alcohol having 1 to 5 carbon atoms. Because of high solubility of the compound obtained by the step (4), ethanol as the alcohol is particularly preferably used.

The reducing agent used in the step (5) is preferably at least one type selected from the group consisting of alkali metal salts of borohydride compounds such as sodium borohydride and lithium borohydride; alkaline earth metal salts of borohydride compounds such as magnesium borohydride and calcium borohydride; and aluminum hydride salts such as lithium aluminum hydride and sodium aluminum hydride. Among them, sodium borohydride is particularly preferred because of easy availability and handleability.

For example, in the case of carrying out the step (5) on the final product in formula (i) represented in the step (4), the compound represented by formula (ii) is generated. x in formula (ii) is the same as defined for formula (i).

HO—CH₂—CF₂—O—(CF₂CF₂O)ₓ₋₂—CF₂—CH₂—OH (ii)

### EXAMPLES

The present invention will be specifically described below based on Examples, but the present invention is in no way limited to these Examples.

### <Nmr Measurement>

The number-average molecular weight by NMR measurement is the value measured by 1H-NMR and ¹⁹F-NMR using an AVANCEIII 400 manufactured by Bruker Biospin K. K. In NMR (nuclear magnetic resonance) measurements, a sample was diluted into d-chloroform or d-acetone solvent and used for the measurements. A 1H-NMR chemical shift reference was the tetramethylsilane peak at 0.0 ppm and a ¹⁹F-NMR chemical shift reference was the hexafluorobenzene peak at −164.7 ppm.

In Examples 1 to 6 and Comparative Examples 1 to 3, the average degree of polymerization and the number-average molecular weight (Mn) of each compound were calculated based on the following NMR measurement results.

HO—(CH₂CH₂CH₂O)ᵤ—H wherein u represents an average degree of polymerization.

¹H-NMR (CDCl₃): δ [ppm]=3.7 to 3.9 (4H), 3.4 to 3.6 (4 (u-1)H), 2.0 to 3.0 (2H), 1.7 to 1.9 (2 uH) CH₃—(C═O)—O—(CH₂CH₂CH₂O)ₙ—(C═O)—CH₃ wherein n represents an average degree of polymerization.

¹H-NMR (CDCl₃): δ [ppm]=4.0 to 4.3 (4H), 3.2 to 3.3 (4 (n-1)H), 2.0 (6H), 1.7 to 1.9 (2 nH) CH₃O—(C═O)—CF₂CF₂O—(CF₂CF₂CF₂O)ₙ—CF₂CF₂—(C═O)—OCH₃ wherein m is an average degree of polymerization.

¹H-NMR (acetone-D₆): δ [ppm]=4.07 (6H)

¹⁹F-NMR (acetone-D₆): δ [ppm]=−84.2 to −84.3 (4mF), −86.1 to −86.4 (4F), −122.5 to −122.8 (4F), −129.8 to −130.2 (2mF) HO—CH₂—CF₂CF₂O—(CF₂CF₂CF₂O)ₘ—CF₂CF₂—CH₂—OH wherein m′ represents an average degree of polymerization.

¹H-NMR (acetone-D₆): δ [ppm]=5.1 to 5.2 (2H), 4.0 to 4.1 (4H)

¹⁹F-NMR (acetone-D₆): δ [ppm]=−84.2 to −84.8 (4m′F), −86.4 to −87.3 (4F), −126.3 to −126.7 (4F), −129.8 to −130.2 (2m′F)

In Examples 7 and 8, the average degree of polymerization and the number-average molecular weight (Mn) of each compound were calculated based on the following NMR measurement results.

CH₃—(C═O)—O—(CH₂CH₂O)ₙ—(C═O)—CH₃ wherein n represents an average degree of polymerization.

¹H-NMR (CDCl₃): δ [ppm]=4.2 to 4.3 (4H), 3.6 to 3.7 (4 (n-1)H), 2.0 (6H)

CH₃O—(C═O)—CF₂O—(CF₂CF₂O)ₘ—CF₂—(C═O)—OCH₃ wherein m represents an average degree of polymerization.

¹H-NMR (acetone-D₆): δ [ppm]=4.06 (6H)

¹⁹F-NMR (acetone-DF): 5 [ppm]=−78.5 to −78.6 (4F), −88.0 to −89.8 (4mF)

### <Gpc Measurement>

50 mg of a sample was dissolved in 1 mL of tetrahydrofuran and used as a measurement sample. GPC measurement conditions are shown below. “EasiVial PEG” manufactured by Agilent Technology Inc., was used as a standard material, and molecular weight distribution data (GPC chart) was obtained based on a calibration curve prepared using the standard material.
Apparatus name: an HPLC Prominence UFLC manufactured by SHIMADZU CORPORATION
Column: Shodex (registered trademark) KF-402HQ (1 column) and KF-401HQ (3 columns), manufactured by Showa Denko K. K., connected in series and used.
Column temperature: 30° C.
Mobile phase: tetrahydrofuran
Injection volume: 1 μL
Flow rate: 0.2 mL/min
Detector: RI

Mw/Mn of the compound to be adjusted in the step (1) and Mw/Mn of the raw material in the step (2) were obtained from the GPC measurement results described above. Mn in Mw/Mn is the value obtained by GPC measurement, not by NMR.

Proportions of low molecular weight components (components with degrees of polymerization of 1 and 2) and high molecular weight components (components with degrees of polymerization of (average degree of polymerization+4) or higher), contained in the raw materials of the step (2), were calculated from the GPC measurement results described above. The total peak area of a raw material compound is an area of the entire peak excluding a peak of an impurity. Peaks of each degree of polymerization were vertically divided at their minimum and inflection points.

A proportion of low molecular weight component (%)=(Sum of peak areas of components with degrees of polymerization of 1 and 2)/(Total peak area of raw material compound)×100

A proportion of high molecular weight component (%)=(Sum of peak areas of components with degrees of polymerization of (average degree of polymerization+4) or higher)/(Total peak area of raw material compound)×100

For some peaks of high molecular weight components that were hard to separate, the maximum value of a proportion of components with degrees of polymerization of (average degree of polymerization+4) or higher, was calculated based on a proportion of components with degrees of polymerization of (average degree of polymerization+3) or higher or a proportion of components with degrees of polymerization of (average degree of polymerization+2) or higher.

### <Calculation of Collection Ratios in Steps (2) to (4)>

Collection ratio (%)=(Mass of product in step (4) (g) /Theoretical value of number-average molecular weight of product in step (4) (g/mol))/(Mass of raw material in the step (2) (g)/Number-average molecular weight of raw material in the step (2) (g/mol))×100

### <Theoretical Values of Average Degree of Polymerization and Number-Average Molecular Weight>

In the case of the raw material (fluorination raw material) of the step (2) being CH₃—(C═O)—O—(CH₂CH₂CH₂O)ₙ—(C═O)—CH₃, when its average degree of polymerization is n, a number-average molecular weight of the fluorination raw material is 58.08 n+102.09. A theoretical value of an average degree of polymerization of the product of the step (4) is represented as “n-2”, and a theoretical value of a number-average molecular weight of the product of the step (4) is represented as 166.02 n+2.08.

In the case of the raw material (fluorination raw material) in the step (2) being CH₃—(C═O)—O—(CH₂CH₂O)ₙ—(C═O)—CH₃, when its average degree of polymerization is n, a number-average molecular weight of the fluorination raw material is 44.05 n+102.09. A theoretical value of an average degree of polymerization of the product of the step (4) is represented as “n-2”, and a theoretical value of a number-average molecular weight of the product of the step (4) is 116.01 n+2.08.

### Example 1

### Step (1)

HO—(CH₂CH₂CH₂O)ᵤ—H wherein u represents an average degree of polymerization, synthesized by sequential polymerization, was reacted with acetyl chloride to acetylate hydroxyl groups at both ends. The resulting CH₃—(C═O)—O—(CH₂CH₂CH₂O)_{w}—(C═O)—CH₃ wherein w represents an average degree of polymerization and w=4.51, was used as the compound to be adjusted in the step (1). Distillation under reduced pressure (200 to 240° C., 22 to 27 Pa) was performed on 118 g of the compound to be adjusted. Components with degrees of polymerization of 3 to 7 were fractionated to obtain CH—(C═O)—O—(CH₂CH₂CH₂O)ₙ—(C═O)—CH₃ as a raw material compound with an adjusted molecular weight distribution, wherein n represents an average degree of polymerization, n=4.06 and Mn of 338 (collection ratio (mass ratio) of 33%).

### Step (2)

A 5 L autoclave was introduced with 3,100 mL of tetrachlorohexafluorobutane (sometimes also referred to as “HFTCB”) and then sealed. An operation of introducing a nitrogen gas to the autoclave until inner pressure thereof reached 0.3 MPa (gauge pressure), and slowly releasing the inner pressure to normal pressure was performed 10 times, to purge the autoclave. 39 g of CH₃—(C═O)—O—(CH₂CH₂CHO)ₙ—(C═O)—CH; obtained in the step (1) wherein n represents an average degree of polymerization, n=4.06, and Mn of 338, was dissolved in 14.9 mL of HFTCB to prepare a raw material solution. A fluorine gas and a nitrogen gas were distributed at 588 mL/min and 4,600 mL/min, respectively, and while cooled to an internal temperature of 25° C., the raw material solution was introduced into distribution at a flow rate of 0.23 g/min on a solution mass basis, to carry out a fluorination reaction.

### Step (3)

1.87 g of hexafluorobenzene (C(FE) was dissolved in 73 mL of HFTCB to prepare a C₆F₆ solution. An inner temperature in the autoclave was adjusted to 25 to 30° C., and the C₆F₆ solution was introduced into distribution at a flow rate of 0.83 g/min on a solution mass basis while distributing a fluorine gas and a nitrogen gas at 150 mL/min and 1,350 mL/min, respectively.

### Step (4)

Following completion of introduction of the C₆F₆ in the step (3), the distribution of fluorine gas and nitrogen gas was continued for 10 minutes, the distribution of fluorine gas was then terminated, and a nitrogen gas was distributed at 1,350 mL/min for 1 hour to purge the autoclave. While distributing the nitrogen gas, 74 g of methanol was introduced.

A reaction solution collected from the autoclave was washed with sodium carbonate water. After separation of the solution into two layers, an organic solvent layer was collected, dried with sodium sulfate and sodium fluoride, and then the solid was filtered off. The solvent was distilled off by using an evaporator to obtain 75 g of a product (collection ratio of 96%). As a result of having analyzed the obtained product by ⁹F-NMR, it was confirmed to be CH₃₀—(C═O)—CF₂CF₂O—(CF₂CF₂CF₂O)ₘ—CF₂CF₂—(C═O)—OCH₃ wherein m represents an average degree of polymerization, m=2.18, and Mn of 696.

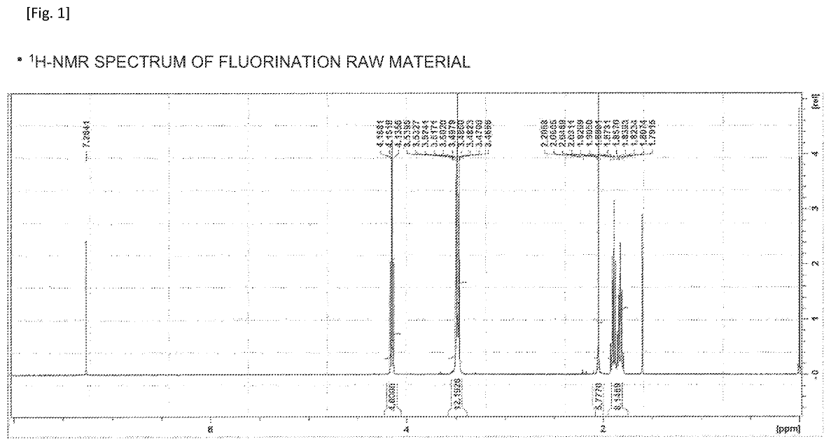

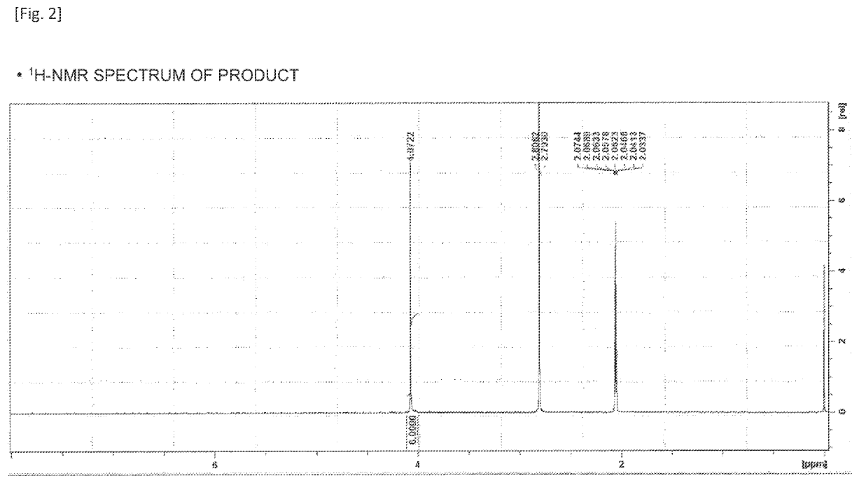

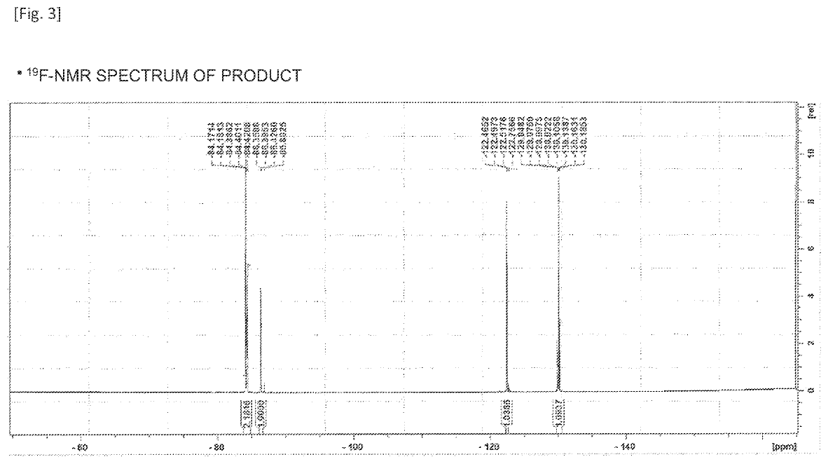

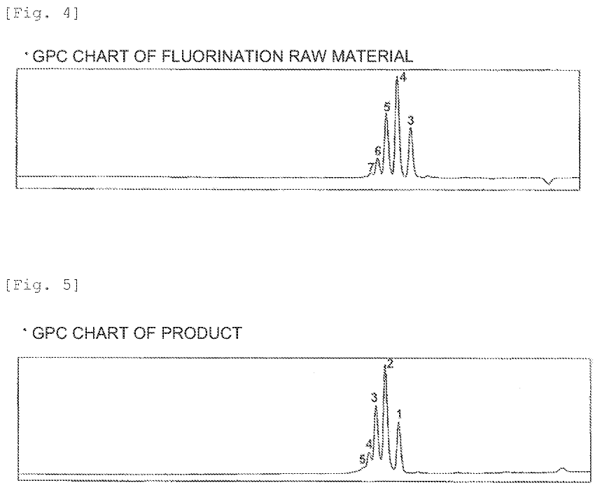

The ¹H-NMR spectrum of the fluorination raw material in Example 1 is shown in FIG. 1, the ¹H-NMR spectrum of the product of the step (4) in Example 1 is shown in FIG. 2, the ¹⁹F-NMR spectrum of the product of the step (4) in Example 1 is shown in FIG. 3, the GPC chart of the fluorination raw material in Example 1 is shown in FIG. 4, and the GPC chart of the product of the step (4) in Example 1 is shown in FIG. 5. The numerical values shown in the GPC charts in FIG. 4 and FIG. 5 represent degrees of polymerization of the components corresponding to each peak.

### Example 2

### Step (1)

HO—(CH₂CH₂CH₂O)ᵤ—H wherein u represents an average degree of polymerization, synthesized by sequential polymerization, was reacted with acetyl chloride to acetylate hydroxyl groups at both ends. The resulting CH₃—(C═O)—O—(CH₂CH₂CH₂O)_{w}—(C═O)—CH₃ wherein w represents an average degree of polymerization, and w=4.70, was used as the compound to be adjusted in the step (1). 99 g of the compound to be adjusted was fractionated in two times by preparative chromatography (500 g of silica, normal hexane/ethyl acetate=90/10 to 0/100 (volume ratio)). Components with degrees of polymerization of 3 to 7 were fractionated to obtain CH₃—(C═O)—O—(CH₂CH₂CH₂O)ₙ—(C═O)—CH₃ as a raw material compound with an adjusted molecular weight distribution, wherein n represents an average degree of polymerization, n=4.59, and Mn of 369 (collection ratio (mass ratio) of 49%).

### Step (2)

A 5 L autoclave was introduced with 3,100 mL of HFTCB and then sealed. An operation of introducing a nitrogen gas to the autoclave until inner pressure thereof reached 0.3 MPa, and slowly releasing the inner pressure to normal pressure was performed 10 times, to purge the autoclave. 46 g of CH₃—(C═O)—O—(CH₂CH₂CH₂O), —(C═O)—CH₃ obtained in the step (1) wherein n represents an average degree of polymerization, n=4.59, and Mn of 369, was dissolved in 17.5 mL of HFTCB to prepare a raw material solution. A fluorine gas and a nitrogen gas were distributed at 588 mL/min and 4,600 mL/min, respectively, and while cooled to an internal temperature of 25° C., the raw material solution was introduced into distribution at a flow rate of 0.23 g/min on a solution mass basis, to carry out a fluorination reaction.

### Step (3)

1.87 g of C₆F₆ was dissolved in 73 mL of HFTCB to prepare a C₆F₆ solution. An inner temperature in the autoclave was adjusted to 25 to 30° C., and the C₆F₆ solution was introduced into distribution at a flow rate of 0.83 g/min on a solution mass basis while distributing a fluorine gas and a nitrogen gas at 150 mL/min and 1,350 mL/min, respectively.

### Step (4)

Following completion of introduction of the C₆F₆ in the step (3), the distribution of fluorine gas and nitrogen gas was continued for 10 minutes, the distribution of fluorine gas was then terminated, and a nitrogen gas was distributed at 1,350 mL/min for 1 hour to purge the autoclave. While distributing the nitrogen gas, 80 g of methanol was introduced.

A reaction solution collected from the autoclave was washed with sodium carbonate water. After separation of the solution into two layers, an organic solvent layer was collected, dried with sodium sulfate and sodium fluoride, and then the solid was filtered off. The solvent was distilled off by using an evaporator to obtain 92 g of a product (collection ratio of 97%). As a result of having analyzed the obtained product by ¹⁹F-NMR, it was confirmed to be CH₃O—(C═O)—CF₂CF₂O—(CF₂CF₂CF₂O)ₘ—CF₂CF₂—(C═O)—OCH₃ wherein m represents an average degree of polymerization, m=2.68, and Mn of 779.

### Step (5)

A reduction reaction of CH₃O—(C═O)—CF₂CF₂O—(CF₂CF₂CF₂O)ₘ—CF₂CF₂—(C═O)—OCH₃ obtained in the step (4) was carried out by the following method.

An eggplant flask was charged with 335 g of ethanol, cooled to 0° C., and then charged with 5.7 g of sodium borohydride. While cooling the solution to 0° C., 89.8 g of CH₃O—(C═O)—CF₂CF₂O—(CF₂CF₂CF₂O)ₘ—CF₂CF₂—(C═O)—OCH₃ wherein m represents an average degree of polymerization, m=2.68, and Mn of 779, was added dropwise at 2.7 g/min, and was washed in with 38 g of ethanol. The reaction was allowed to continue for 4.5 hours while the eggplant flask was brought to room temperature.

After completion of reaction, 38 mL of 4M hydrochloric acid was added dropwise to confirm that a pH of the solution was 3, and then a sodium bicarbonate aqueous solution (192 g of sodium bicarbonate, 200 mL of water) was added to confirm that a pH of the solution was 8. Ethanol was distilled off by an evaporator, and following addition of 250 mL of water, an aqueous layer was extracted in three times with 750 mL of ethyl acetate. After having dried the solution with magnesium sulfate, a solid was filtered off, and the solvent was distilled off by an evaporator to obtain 87.7 g (collection ratio of 106%) of a product HO—CH₂—CF₂CF₂O—(CF₂CF₂CF₂₀)_{m′}—CF₂CF₂—CH₂—OH wherein m′ represents an average degree of polymerization.

### Example 3

### Step (1)

HO—(CH₂CH₂CH₂O)ᵤ—H wherein u represents an average degree of polymerization, synthesized by sequential polymerization, was reacted with acetyl chloride to acetylate hydroxyl groups at both ends. The resulting CH₃—(C═O)—O—(CH₂CH₂CH₂O)_{w}—(C═O)—CH₃ wherein w represents an average degree of polymerization and w=3.13, was used as the compound to be adjusted in the step (1). Distillation under reduced pressure (110 to 160° C., 106 to 200 Pa, with a filler DIXON PACKING having an outer diameter of 6 mm, manufactured by NIHON MESH KOGYO., LTD) was performed on 105 g of the compound to be adjusted. Components with degrees of polymerization of 1 and 2 were distilled off to obtain CH₃—(C═O)—O—(CH₂CH₂CH₂O)ₙ—(C═O)—CH₃ as a raw material compound with an adjusted molecular weight distribution wherein n represents an average degree of polymerization, n=4.72, and Mn of 376 (collection ratio (mass ratio) of 66%).

### Step (2)

A 5 L autoclave was introduced with 3,100 mL of HFTCB and then sealed. An operation of introducing a nitrogen gas to the autoclave until inner pressure thereof reached 0.3 MPa, and slowly releasing the inner pressure to normal pressure was performed 10 times, to purge the autoclave. 34 g of CH₃—(C═O)—O—(CH₂CH₂CH₂O)ₙ—(C═O)—CH₃ obtained in the step (1) wherein n represents an average degree of polymerization, n=4.72, and Mn of 376, was dissolved in 12.9 mL of HFTCB to prepare a raw material solution. A fluorine gas and a nitrogen gas were distributed at 588 mL/min and 4,600 mL/min, respectively, and while cooled to an internal temperature of 25° C., the raw material solution was introduced into distribution at a flow rate of 0.23 g/min on a solution mass basis, to carry out a fluorination reaction.

### Step (3)

1.87 g of C₆F₆ was dissolved in 73 mL of HFTCB to prepare a C₆F₆ solution. An inner temperature in the autoclave was adjusted to 25 to 30° C., and the C₆F₆ solution was introduced into distribution at a flow rate of 0.83 g/min on a solution mass basis while distributing a fluorine gas and a nitrogen gas at 150 mL/min and 1,350 mL/min, respectively.

### Step (4)

Following completion of introduction of the C₆F₆ in the step (3), the distribution of fluorine gas and nitrogen gas was continued for 10 minutes, the distribution of fluorine gas was then terminated, and a nitrogen gas was distributed at 1,350 mL/min for 1 hour to purge the autoclave. While distributing the nitrogen gas, 47 g of methanol was introduced.

A reaction solution collected from the autoclave was washed with sodium carbonate water. After separation of the solution into two layers, an organic solvent layer was collected, dried with sodium sulfate and sodium fluoride, and then the solid was filtered off. The solvent was distilled off by using an evaporator to obtain 65 g of a product (collection ratio of 91%). As a result of having analyzed the obtained product by ¹⁹F-NMR, it was confirmed to be CH₃O—(C═O)—CF₂CF₂O—(CF₂CF₂CF₂O)ₘ—CF₂CF₂—(C═O)—OCH₃ wherein m represents an average degree of polymerization, m=2.97, and Mn of 827.

### Example 4

### Step (1)

HO—(CH₂CH₂CH₂O)ᵤ—H synthesized by sequential polymerization wherein u represents an average degree of polymerization, u=4.47, and Mn of 278, was used as the first compound to be adjusted in the step (1). 175 g of the compound to be adjusted and 175 g of water were mixed at room temperature and cooled to 5° C. to precipitate a solid. High molecular weight components were removed by filtrating off the precipitated solid, and the residue was obtained by distilling off water from the filtrate (collection ratio (mass ratio) of 71%). HO—(CH₂CH₂CH₂O)ᵥ—H obtained as the residue wherein v represents an average degree of polymerization, v=3.90, and Mn of 245, had Mw/Mn of 1.26.

HO—(CH₂CH₂CH₂O)ᵥ—H was then reacted with acetyl chloride to acetylate hydroxyl groups at both ends.

The resulting CH₃—(C═O)—O—(CH₂CH₂CH₂O)_{w}—(C═O)—CH₃ wherein w represents an average degree of polymerization, w=3.90, and Mn of 329 (calculated values based on the degree of polymerization before acetylation), was used as the second compound to be adjusted in the step (1). Distillation under reduced pressure (110 to 180° C., 120 to 173 torr (16,000 to 23,000 Pa) with a filler DIXON PACKING having an outer diameter of 6 mm, manufactured by NIHON MESH KOGYO., LTD), was performed on 161 g of the compound to be adjusted. Components with degrees of polymerization of 1 and 2 were distilled off to obtain CH₃—(C═O)—O—(CH₂CH₂CH₂O)ₙ—(C═O)—CH₃ as a raw material compound with an adjusted molecular weight distribution wherein n represents an average degree of polymerization, n=4.87, and Mn of 385 (collection ratio (mass ratio) of 79%).

### Step (2)

A 5 L autoclave was introduced with 3,100 mL of HFTCB and then sealed. An operation of introducing a nitrogen gas to the autoclave until inner pressure thereof reached 0.3 MPa, and slowly releasing the inner pressure to normal pressure was performed 10 times, to purge the autoclave. 63 g of CH₃—(C═O)—O—(CH₂CH₂CH₂O)ₙ—(C═O)—CH₃ obtained in the step (1) wherein n represents an average degree of polymerization, n=4.87, and Mn of 385, was dissolved in 24.0 mL of HFTCB to prepare a raw material solution. A fluorine gas and a nitrogen gas were distributed at 588 mL/min and 4,600 mL/min, respectively, and while cooled to an internal temperature of 25° C., the raw material solution was introduced into distribution at a flow rate of 0.23 g/min on a solution mass basis, to carry out a fluorination reaction.

### Step (3)

1.87 g of C₆F₆ was dissolved in 73 mL of HFTCB to prepare a C₆F₆ solution. An inner temperature in the autoclave was adjusted to 25 to 30° C., and the C₆F₆ solution was introduced into distribution at a flow rate of 0.83 g/min on a solution mass basis while distributing a fluorine gas and a nitrogen gas at 150 mL/min and 1,350 mL/min, respectively.

### Step (4)

Following completion of introduction of the C₆F₆ in the step (3), the distribution of fluorine gas and nitrogen gas was continued for 10 minutes, the distribution of fluorine gas was then terminated, and a nitrogen gas was distributed at 1,350 mL/min for 1 hour to purge the autoclave. While distributing the nitrogen gas, 105 g of methanol was introduced.

A reaction solution collected from the autoclave was washed with sodium carbonate water. After separation of the solution into two layers, an organic solvent layer was collected, dried with sodium sulfate and sodium fluoride, and then the solid was filtered off. The solvent was distilled off by using an evaporator to obtain 127 g of a product (collection ratio of 96%). As a result of having analyzed the obtained product by ¹⁹F-NMR, it was confirmed to be CH₃O—(C═O)—CF₂CF₂O—(CF₂CF₂CF₂O)ₘ—CF₂CF₂—(C═O)—OCH₃ wherein m represents an average degree of polymerization, m=3.09, and Mn of 847.

### Example 5

### Step (1)

HO—(CH₂CH₂CHO)ᵤ—H wherein u represents an average degree of polymerization, synthesized by sequential polymerization, was reacted with acetyl chloride to acetylate hydroxyl groups at both ends. The resulting CH₃—(C═O)—O—(CH₂CH₂CH₂O)_{w}—(C═O)—CH₃ wherein w represents an average degree of polymerization and w=5.39, was used as the compound to be adjusted in the step (1). 100 g of the compound to be adjusted was fractionated in two times by preparative chromatography (500 g of silica, normal hexane/ethyl acetate=90/10 to 0/100 (volume ratio)). Components with degrees of polymerization of 4 to 8 were fractionated to obtain CH₃—(C═O)—O—(CH₂CH₂CH₂O)ₙ—(C═O)—CH₃ as a raw material compound with an adjusted molecular weight distribution wherein n represents an average degree of polymerization, n=5.66, and Mn of 431 (collection ratio (mass ratio) of 51%).

### Step (2)

A 5 L autoclave was introduced with 3,100 mL of HFTCB and then sealed. An operation of introducing a nitrogen gas to the autoclave until inner pressure thereof reached 0.3 MPa, and slowly releasing the inner pressure to normal pressure was performed 10 times, to purge the autoclave. 49 g of CH₃—(C═O)—O—(CH₂CH₂CH₂O)ₙ—(C═O)—CH₃ obtained in the step (1) wherein n represents an average degree of polymerization, n=5.66, and Mn=431, was dissolved in 18.8 mL of HFTCB to prepare a raw material solution. A fluorine gas and a nitrogen gas were distributed at 588 mL/min and 4,600 mL/min, respectively, and while cooled to an internal temperature of 25° C., the raw material solution was introduced into distribution at a flow rate of 0.22 g/min on a solution mass basis, to carry out a fluorination reaction.

### Step (3)

1.87 g of C₆F₆ was dissolved in 73 mL of HFTCB to prepare a C₆F₆ solution. An inner temperature in the autoclave was adjusted to 25 to 30° C., and the C₆F₆ solution was introduced into distribution at a flow rate of 0.83 g/min on a solution mass basis while distributing a fluorine gas and a nitrogen gas at 150 mL/min and 1,350 mL/min, respectively.

### Step (4)

Following completion of introduction of the C₆F₆ in the step (3), the distribution of fluorine gas and nitrogen gas was continued for 10 minutes, and then distribution of fluorine gas was terminated, and a nitrogen gas was distributed at 1,350 mL/min for 1 hour to purge the autoclave. While distributing the nitrogen gas, 73 g of methanol was introduced.

A reaction solution collected from the autoclave was washed with sodium carbonate water. After separation of the solution into two layers, an organic solvent layer was collected, dried with sodium sulfate and sodium fluoride, and then the solid was filtered off. The solvent was distilled off by using an evaporator to obtain 106 g of a product (collection ratio of 99%). As a result of having analyzed the obtained product by ¹⁹F-NMR, it was confirmed to be CHO—(C═O)—CF₂CF₂O—(CF₂CF₂CF₂O)ₘ—CF₂CF₂—(C═O)—OCH₃ wherein m represents an average degree of polymerization, m=3.82, and Mn of 968.

### Example 6

### Step (1)

HO—(CH₂CH₂CH₂O)_{L}—H wherein u represents an average degree of polymerization, synthesized by sequential polymerization, was reacted with acetyl chloride to acetylate hydroxyl groups at both ends. The resulting CH₂—(C═O)—O—(CH₂CH₂CH₂O)_{w}—(C═O)—CH₃ wherein w represents an average degree of polymerization and w=7.35, was used as the compound to be adjusted in the step (1). 117 g of the compound to be adjusted was fractionated in two times by preparative chromatography (500 g of silica, normal hexane/ethyl acetate=90/10 to 0/100 (volume ratio)). Components with degrees of polymerization of 5 to 9 were fractionated to obtain CH₃—(C═O)—O—(CH₂CH₂CH₂O)ₙ—(C═O)—CH₃ as a raw material compound with an adjusted molecular weight distribution wherein n represents an average degree of polymerization, n=6.88, and Mn of 502 (collection ratio (mass ratio) of 22%).

### Step (2)

A 5 L autoclave was introduced with 3,100 mL of HFTCB and then sealed. An operation of introducing a nitrogen gas to the autoclave until inner pressure thereof reached 0.3 MPa, and slowly releasing the inner pressure to normal pressure was performed 10 times, to purge the autoclave. 25 g of CH₃—(C═O)—O—(CH₂CH₂CH₂O)ₙ—(C═O)—CH₃ obtained in the step (1) wherein n represents an average degree of polymerization, n=6.88, and Mn=502, was dissolved in 9.7 mL of HFTCB to prepare a raw material solution. A fluorine gas and a nitrogen gas were distributed at 588 mL/min and 4,600 mL/min, respectively, and while cooled to an internal temperature of 25° C., the raw material solution was introduced into distribution at a flow rate of 0.22 g/min on a solution mass basis, to carry out a fluorination reaction.

### Step (3)

1.87 g of C₆F₆ was dissolved in 73 mL of HFTCB to prepare a C₆F₆ solution. An inner temperature in the autoclave was adjusted to 25 to 30° C., and the C₆F₆ solution was introduced into distribution at a flow rate of 0.83 g/min on a solution mass basis while distributing a fluorine gas and a nitrogen gas at 150 mL/min and 1,350 mL/min, respectively.

### Step (4)

Following completion of introduction of the C₆F₆ in the step (3), the distribution of fluorine gas and nitrogen gas was continued for 10 minutes, the distribution of fluorine gas was then terminated, and a nitrogen gas was distributed at 1,350 mL/min for 1 hour to purge the autoclave. While distributing the nitrogen gas, 32 g of methanol was introduced.

A reaction solution collected from the autoclave was washed with sodium carbonate water. After separation of the solution into two layers, an organic solvent layer was collected, dried with sodium sulfate and sodium fluoride, and then the solid was filtered off. The solvent was distilled off by using an evaporator to obtain 56 g of a product (collection ratio of 98%). As a result of having analyzed the obtained product by ¹⁹F-NMR, it was confirmed to be CH₃O—(C═O)—CF₂CF₂O—(CF₂CF₂CF₂O)ₘ—CF₂CF₂—(C═O)—OCH₃ wherein m represents an average degree of polymerization, m=5.06, and Mn of 1174.

### Example 7

### Step (1)

HO—(CH₂CH₂O)ᵤ—H wherein u represents an average degree of polymerization, which was a commercially available product, was reacted with acetyl chloride to acetylate hydroxyl groups at both ends. The resulting CH₃O—(C═O)—O—(CH₂CH₂O)_{w}—(C═O)—CH₃ wherein w represents an average degree of polymerization, was used as the compound to be adjusted in the step (1). 62 g of the compound to be adjusted was fractionated by preparative chromatography (400 g of silica, normal hexane/ethyl acetate/isopropyl alcohol=80/20/0 to 0/100/0 to 0/80/20 (volume ratio)). Components with degrees of polymerization of 5 to 8 were fractionated to obtain CH₃O—(C═O)—O—(CH₂CH₂O)ₙ—(C═O)—CH₃ as a raw material compound with an adjusted molecular weight distribution wherein n represents an average degree of polymerization, n=6.46, and Mn of 387 (collection ratio (mass ratio) of 49%).

### Step (2)

A 5 L autoclave was introduced with 3,100 mL of HFTCB and then sealed. An operation of introducing a nitrogen gas to the autoclave until inner pressure thereof reached 0.3 MPa, and slowly releasing the inner pressure to normal pressure was performed 10 times, to purge the autoclave. 42 g of CH₃O—(C═O)—O—(CH₂CH₂O)ₙ—(C═O)—CH₃ obtained in the step (1) wherein n represents an average degree of polymerization, n=6.46, and Mn of 387, was dissolved in 15.9 mL of HFTCB to prepare a raw material solution. A fluorine gas and a nitrogen gas were distributed at 588 mL/min and 4,600 mL/min, respectively, and while cooled to an internal temperature of 25° C., the raw material solution was introduced into distribution at a flow rate of 0.25 g/min on a solution mass basis, to carry out a fluorination reaction.

### Step (3)

1.87 g of C₆F₆ was dissolved in 73 mL of HFTCB to prepare a C₆F₆ solution. An inner temperature in the autoclave was adjusted to 25 to 30° C., and the C₆F₆ solution was introduced into distribution at a flow rate of 0.83 g/min on a solution mass basis while distributing a fluorine gas and a nitrogen gas at 150 mL/min and 1,350 mL/min, respectively.

### Step (4)

Following completion of introduction of the C₆F₆ in the step (3), the distribution of fluorine gas and nitrogen gas was continued for 10 minutes, the distribution of fluorine gas was then terminated, and a nitrogen gas was distributed at 1,350 mL/min for 1 hour to purge the autoclave. While distributing the nitrogen gas, 69 g of methanol was introduced.

A reaction solution collected from the autoclave was washed with sodium carbonate water. After separation of the solution into two layers, an organic solvent layer was collected, dried with sodium sulfate and sodium fluoride, and then the solid was filtered off. The solvent was distilled off by using an evaporator to obtain 71 g of a product (collection ratio of 87%). As a result of having analyzed the obtained product by ¹⁹F-NMR, it was confirmed to be CH₃O—(C═O)—CF₂O—(CF₂CF₂O)ₙ—CF₂—(C═O)—OCH₃ wherein m represents an average degree of polymerization, m=4.48, and Mn of 754.

### Example 8

### Step (1)

HO—(CH₂CH₂O)ᵤ—H wherein u represents an average degree of polymerization, which was a commercially available product, was reacted with acetyl chloride to acetylate hydroxyl groups at both ends. The resulting CH₃O—(C═O)—O—(CH₂CH₂O)_{w}—(C═O)—CH₃ wherein w represents an average degree of polymerization, w=7.65, and Mn of 439, was used as the compound to be adjusted in the step (1). 62 g of the compound to be adjusted was fractionated by preparative chromatography (500 g of silica, normal hexane/ethyl acetate=90/10 to 0/100 (volume ratio)). Components with degrees of polymerization of 6 to 9 were fractionated to obtain CH₃O—(C═O)—O—(CH₂CH₂O)ₙ—(C═O)—CH₃ as a raw material compound with an adjusted molecular weight distribution wherein n represents an average degree of polymerization, n=7.50, and Mn of 433 (collection ratio (mass ratio) of 48%).

### Step (2)

A 5 L autoclave was introduced with 3,100 mL of HFTCB and then sealed. An operation of introducing a nitrogen gas to the autoclave until inner pressure thereof reached 0.3 MPa, and slowly releasing the inner pressure to normal pressure was performed 10 times, to purge the autoclave. 29 g of CH₃O—(C═O)—O—(CH₂CH₂O)ₙ—(C═O)—CH₃ obtained in the step (1) wherein n represents an average degree of polymerization, n=7.50, and Mn of 433, was dissolved in 149.6 mL of HFTCB to prepare a raw material solution. A fluorine gas and a nitrogen gas were distributed at 588 mL/min and 4,600 mL/min, respectively, and while cooled to an internal temperature of 25° C., the raw material solution was introduced into distribution at a flow rate of 1.45 g/min on a solution mass basis, to carry out a fluorination reaction.

### Step (3)

1.87 g of C₆F₆ was dissolved in 73 mL of HFTCB to prepare a C₆F₆ solution. An inner temperature in the autoclave was adjusted to 25 to 30° C., and the C₆F₆ solution was introduced into distribution at a flow rate of 0.83 g/min on a solution mass basis while distributing a fluorine gas and a nitrogen gas at 150 mL/min and 1,350 mL/min, respectively.

### Step (4)

Following completion of introduction of the C₆F₆ in the step (3), the distribution of fluorine gas and nitrogen gas was continued for 10 minutes, the distribution of fluorine gas was then terminated, and a nitrogen gas was distributed at 1,350 mL/min for 1 hour to purge the autoclave. While distributing the nitrogen gas, 43 g of methanol was introduced.

A reaction solution collected from the autoclave was washed with sodium carbonate water. After separation of the solution into two layers, an organic solvent layer was collected, dried with sodium sulfate and sodium fluoride, and then the solid was filtered off. The solvent was distilled off by using an evaporator to obtain 49 g of a product (collection ratio of 84%). As a result of having analyzed the obtained product by ¹⁹F-NMR, it was confirmed to be CH₃O—(C═O)—CF₂O—(CF₂CF₂O)ₙ—CF₂—(C═O)—OCH₃ wherein m represents an average degree of polymerization, m=5.36, and Mn of 856.

### Comparative Example 1

HO—(CH₂CH₂CH₂O)ᵤ—H wherein u represents an average degree of polymerization, synthesized by sequential polymerization, was reacted with acetyl chloride to acetylate hydroxyl groups at both ends. Except that 48 g of the resulting CH₃—(C═O)—O—(CH₂CH₂CH₂O)ₙ—(C═O)—CH₃ wherein n represents an average degree of polymerization, n=4.68 and Mn of 374, was directly subjected to the step (2) as a fluorination raw material without adjustment of molecular weight distribution, the reaction was carried out in the same manner as in the step (2) to the step (4) of Example 1 to obtain 87 g (collection ratio 88%) of a product in the step (4). As a result of having analyzed the obtained product by ¹⁹F-NMR, it was confirmed to be CH₃O—(C═O)—CF₂CF₂O—(CF₂CF₂CF₂O)ₘ—CF₂CF₂—(C═O)—OCH₃ wherein m represents an average degree of polymerization, m=3.94 and Mn of 988.

### Comparative Example 2

HO—(CH₂CH₂CH₂O)ᵤ—H wherein u represents an average degree of polymerization, synthesized by sequential polymerization, was reacted with acetyl chloride to acetylate hydroxyl groups at both ends. Except that 42 g of the resulting CH₃—(C═O)—O—(CH₂CH₂CH₂O)ₙ—(C═O)—CH₃ wherein n represents an average degree of polymerization, n=7.83 and Mn of 557, was directly subjected to the step (2) as a fluorination raw material without adjustment of molecular weight distribution, the reaction was carried out in the same manner as in the step (2) to the step (4) of Example 1 to obtain 63 g (collection ratio 64%) of a product in the step (4). As a result of having analyzed the obtained product by ¹⁹F-NMR, it was confirmed to be CH₃O—(C═O)—CF₂CF₂O—(CF₂CF₂CF₂O)ₙ—CF₂CF₂—(C═O)—OCH₃ wherein m represents an average degree of polymerization, m=4.57 and Mn of 1093.

### Comparative Example 3

HO—(CH₂CH₂CH₂O)ᵤ—H wherein u represents an average degree of polymerization, synthesized by sequential polymerization, was reacted with acetyl chloride to acetylate hydroxyl groups at both ends. Except that 40 g of the resulting CH₃—(C═O)—O—(CH₂CH₂CH₂O)ₙ—(C═O)—CH₃ wherein n represents an average degree of polymerization, n=8.58 and Mn of 600, was directly subjected to the step (2) as a fluorination raw material without adjustment of molecular weight distribution, the reaction was carried out in the same manner as in the step (2) to the step (4) of Example 1 to obtain 82 g (collection ratio 86%) of a product in the step (4). As a result of having analyzed the obtained product by ¹⁹F-NMR, it was confirmed to be CH₃O—(C═O)—CF₂CF₂O—(CF₂CF₂CF₂O)ₙ—CF₂CF₂—(C═O)—OCH₃ wherein m represents an average degree of polymerization, m=5.75 and Mn of 1289.

For each of Examples 1 to 6 and Comparative Examples 1 to 3, Table 1 lists the number-average molecular weight (Mn), average degree of polymerization, and Mw/Mn, of the compound to be adjusted in the step (1); the number-average molecular weight (Mn), average degree of polymerization, Mw/Mn, range of the degree of polymerization, proportion of the low molecular weight component, and proportion of the high molecular weight component, of the raw material in the step (2); the measured values of the number-average molecular weight (Mn) and the average degree of polymerization, and theoretical values of the number-average molecular weight (Mn) and the average degree of polymerization, of the product in the step (4); and the collection ratio in the reactions of steps (2) to (4). In the column of compound to be adjusted in the step (1) of Example 4 in Table 1, the upper row shows the measured values of compound in which the hydroxyl groups were not protected (HO—(CH₂CH₂CH₂O)ᵤ—H) and the lower row shows the measured values of compound in which the hydroxyl groups were protected (the number-average molecular weight and average degree of polymerization were calculated values based on the degree of polymerization before acetylation of the hydroxyl groups).

For each of Examples 7 and 8, Table 2 lists the number-average molecular weight (Mn), average degree of polymerization, Mw/Mn, range of the degree of polymerization, proportion of the low molecular weight component, and proportion of the high molecular weight component, of the raw material in the step (2); the measured values of number-average molecular weight (Mn) and the average degree of polymerization, and the theoretical values of number-average molecular weight (Mn) and the average degree of polymerization, of the product in the step (4); and the collection ratio in the reactions of steps (2) to (4).

TABLE 1

| | | Compound to be adjusted in step (1) | | | |
|---|---|---|---|---|---|
| | | Number-average | Average | | |
| | R₁ in | molecular | degree of | | |
| | formula (X) | weight (Mn) | polymerization | Mw/Mn | Adjustment method in step (1) |
| Example 1 | —CH₂CH₂CH₂— | 364 | 4.51 | 1.35 | The components with degrees of |
| | | | | | polymerization of 3 to 7 were acquired by |
| | | | | | distillation under reduced pressure. |
| Example 2 | —CH₂CH₂CH₂— | 375 | 4.70 | 1.38 | The components with degrees of |
| | | | | | polymerization of 3 to 7 were acquired by |
| | | | | | fractionation chromatography. |
| Example 3 | —CH₂CH₂CH₂— | 284 | 3.13 | 1.41 | The components with degrees of |
| | | | | | polymerization of 1 and 2 were removed by |
| | | | | | distillation under reduced pressure. |
| Example 4 | —CH₂CH₂CH₂— | 278*¹ | 4.47*¹ | 1.35*¹ | The high molecular component was removed |
| | | | | | by crystallization |
| | | 329*² | 3.90*² | 1.23 | The components with degrees of |
| | | | | | polymerization of 1 and 2 were removed by |
| | | | | | distillation under reduced pressure. |
| Example 5 | —CH₂CH₂CH₂— | 415 | 5.39 | 1.41 | The components with degrees of |
| | | | | | polymerization of 4 to 8 were acquired by |
| | | | | | fractionation chromatography. |
| Example 6 | —CH₂CH₂CH₂— | 529 | 7.35 | 1.55 | The components with degrees of |
| | | | | | polymerization of 5 to 9 were acquired by |
| | | | | | fractionation chromatography. |
| Comparative | —CH₂CH₂CH₂— | — | — | — | — |
| Example 1 | | | | | |
| Comparative | —CH₂CH₂CH₂— | — | — | — | — |
| Example 2 | | | | | |
| Comparative | —CH₂CH₂CH₂— | — | — | — | — |
| Example 3 | | | | | |

Raw material of step (2)

| | | | | | Proportion of | |
|---|---|---|---|---|---|---|
| | | | | | low molecular | Proportion of high |
| | Number- | | | | weight component | molecular weight component |
| | average | | | | Components | Components with degrees of |
| | molecular | Average | | Range of | with degrees of | polymerization of |
| | weight | degree of | | degree of | polymerization | (average degree of |
| | (Mn) | polymerization | Mw/Mn | polymerization | of 1 and 2 | polymerization + 4) or higher |
| Example 1 | 338 | 4.06 | 1.04 | 3-7 | 0.87% | Degrees of polymerization of |
| | | | | | | 9 or higher |
| | | | | | | 0.08% |
| Example 2 | 369 | 4.59 | 1.06 | 3-7 | 0.27% | Degrees of polymerization of |
| | | | | | | 9 or higher |
| | | | | | | Less than 1.46% |
| Example 3 | 376 | 4.72 | 1.16 | 3 or higher | 2.25% | Degrees of polymerization of |
| | | | | | | 9 or higher |
| | | | | | | 14.21% |
| Example 4 | 385 | 4.87 | 1.12 | 3 or higher | 0.89% | Degrees of polymerization of |
| | | | | | | 9 or higher |
| | | | | | | 7.66% |
| Example 5 | 431 | 5.66 | 1.05 | 4-8 | 0% | Degrees of polymerization of |
| | | | | | | 10 or higher |
| | | | | | | 0.48% |
| Example 6 | 502 | 6.88 | 1.04 | 5-9 | 0% | Degrees of polymerization of |
| | | | | | | 11 or higher |
| | | | | | | Less than 1.49% |
| Comparative | 374 | 4.68 | 1.44 | 1 or higher | 7.93% | Degrees of polymerization of |
| Example 1 | | | | | | 9 or higher |
| | | | | | | 35.44% |
| Comparative | 557 | 7.83 | 1.58 | 1 or higher | 2.63% | Degrees of polymerization of |
| Example 2 | | | | | | 12 or higher |
| | | | | | | 46.34% |
| Comparative | 600 | 8.58 | 1.43 | 1 or higher | 1.97% | Degrees of polymerization of |
| Example 3 | | | | | | 13 or higher |
| | | | | | | 32.54% |

| | Product of step (4) | | | | |
|---|---|---|---|---|---|
| | Measured value of | | Theoretical value of | | |
| | number-average | Measured value of | number-average | Theoretical value | |
| | molecular weight | average degree of | molecular weight | of average degree | Collection ratio in |
| | (Mn) | polymerization | (Mn) | of polymerization | steps (2) to (4) |
| Example 1 | 696 | 2.18 | 676 | 2.06 | 96% |
| Example 2 | 779 | 2.68 | 764 | 2.59 | 97% |
| Example 3 | 827 | 2.97 | 786 | 2.72 | 91% |
| Example 4 | 847 | 3.09 | 811 | 2.87 | 96% |
| Example 5 | 968 | 3.82 | 942 | 3.66 | 99% |
| Example 6 | 1174 | 5.06 | 1144 | 4.88 | 98% |
| Comparative | 988 | 3.94 | 779 | 2.68 | 88% |
| Example 1 | | | | | |
| Comparative | 1093 | 4.57 | 1302 | 5.83 | 64% |
| Example 2 | | | | | |
| Comparative | 1289 | 5.75 | 1427 | 6.58 | 86% |
| Example 3 | | | | | |

*¹The measured value of the compound HO—(CH₂CH₂CH₂O)ᵤ—H in which the hydroxyl groups were not protected.
*²The calculated value based on the degree of polymerization before acetylation of the hydroxyl groups.

TABLE 2

| | | | Raw material of step (2) | | |
|---|---|---|---|---|---|
| | | | Number- | | |
| | | | average | | |
| | R₁ in | Adjustment | molecular | Average | |
| | formula | Method | weight | degree of | |
| | (X) | in step (1) | (Mn) | polymerization | Mw/Mn |
| Example 7 | —CH₂CH₂— | The components | 387 | 6.46 | 1.02 |
| | | with degrees of | | | |
| | | polymerization of 5 | | | |
| | | to 8 were acquired | | | |
| | | by fractionation | | | |
| | | chromatography. | | | |
| Example 8 | —CH₂CH₂— | The components | 433 | 7.50 | 1.02 |
| | | with degrees of | | | |
| | | polymerization of 6 | | | |
| | | to 9 were acquired | | | |
| | | by fractionation | | | |
| | | chromatography. | | | |

| | Raw material of step (2) | | |
|---|---|---|---|
| | | Proportion of | |
| | | low molecular | Proportion of high |
| | | weight component | molecular weight component |
| | | Components | Components with degrees of |
| | Range of | with degrees of | polymerization of |
| | degree of | polymerization | (average degree of |
| | polymerization | of 1 and 2 | polymerization + 4) or higher |
| Example 7 | 5-8 | 0% | Degrees of polymerization of |
| | | | 11 or higher |
| | | | 0% |
| Example 8 | 6-9 | 0% | Degrees of polymerization of |
| | | | 12 or higher |
| | | | Less than 0.74% |

| | Product of step (4) | | | | |
|---|---|---|---|---|---|
| | Measured value of | | Theoretical value of | | |
| | number-average | Measured value of | number-average | Theoretical value | |
| | molecular weight | average degree of | molecular weight | of average degree | Collection ratio in |
| | (Mn) | polymerization | (Mn) | of polymerization | steps (2) to (4) |
| Example 7 | 754 | 4.48 | 752 | 4.46 | 87% |
| Example 8 | 856 | 5.36 | 872 | 5.50 | 84% |

In each of Examples 1 to 8, the operation of adjusting a molecular weight distribution was performed in the step (1), thereby enabling production of the compound with the number-average molecular weight close to the theoretical value through the reactions in steps (2) to (4) in high yield. In contrast, in each of Comparative Examples 1 to 3, it was found that the difference between the theoretical value and measured value of the number-average molecular weight was large, and the yield of the product in the step (4) was also low.

1. A method for producing a fluorinated polyether, comprising:
a step (1) of performing an operation of adjusting a molecular weight distribution of a polymeric compound having a structural unit (R₁—O) to obtain a raw material compound represented by a formula (X); and
after the step (1), a step (2) of introducing the raw material compound represented by the formula (X), an inert gas, a fluorine gas, and a solvent into a reactor to fluorinate the raw material compound,
R₄—O—(R₁—O)ₓ—R₅ (X)
wherein R₁ represents a divalent hydrocarbon group having 2 to 5 carbon atoms; R₁ in each structural unit represented by (R₁—O) may all be the same, or may be partially or totally different from each other, R₄ and R₅ each independently represent a protecting group for a hydroxyl group; and x represents an average degree of polymerization and is a real number of 2.7 to 15.

2. The method for producing a fluorinated polyether according to claim 1, wherein the step (1) comprises a step (1A) of adjusting a molecular weight distribution of a compound represented by a formula (A):
R₂—O—(R₁—O)ₚ—R₃ (A)
wherein R₁ is the same as defined for the formula (X); R₂ and R₃ each independently represent a protecting group for a hydroxyl group; and p represents an average degree of polymerization and is a real number of 1 or greater.

3. The method for producing a fluorinated polyether according to claim 1, wherein the step (1) comprises a step (1B) of adjusting a molecular weight distribution of a compound represented by a formula (B):
HO—(R₁—O)_{q}—H (B)
wherein R₁ is the same as defined for the formula (X); and q represents an average degree of polymerization and is a real number of 1 or greater.

4. The method for producing a fluorinated polyether according to claim 3, wherein the step (1) comprises, after the step (1B), a step (1C) of protecting a hydroxyl group of a compound obtained by the step (1B).

5. The method for producing a fluorinated polyether according to claim 4, wherein the step (1) comprises, after the step (1C), a step (1D) of adjusting a molecular weight distribution of a compound obtained by the step (1C).

6. The method for producing a fluorinated polyether according to claim 1, wherein the operation of adjusting a molecular weight distribution in the step (1) is at least one operation selected from:
an operation of adjusting a molecular weight distribution of a polymeric compound having the same structural unit as that in the formula (X) so as to become narrower, and
an operation of mixing two or more types of monodisperse polymers of a polymeric compound having the same structural unit as that in the formula (X) to adjust molecular weight distribution.

7. The method for producing a fluorinated polyether according to claim 1, wherein
the operation of adjusting a molecular weight distribution in the step (1) is an operation of adjusting a molecular weight distribution of a polymeric compound having the same structural unit as that in the formula (X) so as to become narrower.

8. The method for producing a fluorinated polyether according to 1, wherein Mw/Mn representing a molecular weight distribution of the raw material compound is 1.30 or smaller.

9. The method for producing a fluorinated polyether according to 1, wherein a total proportion of a compound represented by a formula (X-1) contained in the raw material compound is 5% or less in terms of peak area ratio based on GPC analysis,
R₄—O—(R₁—O)ᵣ—R₅ (X-1)
wherein R₁, R₄, R₅ are the same as defined for the formula (X), and r represents 1 or 2.

10. The method for producing a fluorinated polyether according to claim 1, wherein a total proportion of a compound represented by a formula (X-2) contained in the raw material compound is 15% or less in terms of peak area ratio based on GPC analysis,
R₄—O—(R₁—O)ₛ—R₅ (X-2)
wherein R₁, R₄, and R₅ are the same as defined for the formula (X), and s is an integer and satisfies s≥(an average degree of polymerization x in the formula (X)+4).

11. The method for producing a fluorinated polyether according to claim 1, comprising, after the step (2), a step (3) of introducing a perhalogenated unsaturated hydrocarbon compound into the reactor while distributing an inert gas and a fluorine gas in the reactor.

12. The method for producing a fluorinated polyether according to claim 1, wherein the raw material compound is a compound in which R₄ and R₅ in the formula (X) are acyl groups.

13. A method for producing a compound represented by a formula (Y), comprising a step (4) of reacting the fluorinated polyether obtained by the production method according to claim 1 with an alcohol having 1 to 3 carbon atoms,
R₆O—(C═O)—Rf₂—O—(Rf₁—O)_{y}—Rf₃—(C═O)—OR₇ (Y)
wherein Rf₁ represents a divalent perfluorohydrocarbon group having 2 to 5 carbon atoms; Rf₁ in each structural unit represented by (Rf₁—O) may all be the same, or may be partially or totally different from each other; Rf₂ and Rf₃ each independently represent a perfluorohydrocarbon group having 1 to 4 carbon atoms and are determined according to a structure of a structural unit arranged at an end in the formula (X); R₆ and R₇ each independently represent an alkyl group having 1 to 3 carbon atoms; and y represents an average degree of polymerization and is a real number of 0.7 to 13.

14. A method for producing a compound represented by a formula (Z), comprising a step (5) of reducing esters at both ends of the compound represented by the formula (Y) obtained by the production method according to claim 13,
HO—CH₂—Rf₂—O—(Rf₁—O)_{y}—Rf₃—CH₂—OH (Z)
wherein Rf₁, Rf₂, Rf₃, and y are the same as defined for the formula (Y).
